(19) United States
(12) Patent Application Publication
(10) Pub. No.: US 2025/0215460 A1
(43) Pub. Date: Jul. 3, 2025

(54) MICROBIAL CONSORTIUM FOR THE CONVERSION OF CARBOHYDRATES

(71) Applicant: Ohio State Innovation Foundation, Columbus, OH (US)

(72) Inventor: Shang-Tian YANG, Dublin, OH (US)

(21) Appl. No.: 19/066,871

(22) Filed: Feb. 28, 2025

Related U.S. Application Data
(63) Continuation of application No. PCT/US2023/026619, filed on Jun. 29, 2023.
(60) Provisional application No. 63/401,943, filed on Aug. 29, 2022.

(51) Int. Cl. C12P 7/16; C12N 1/20; C12N 9/02; C12N 9/04; C12R 1/145; C12R 1/225; C12R 1/46

(52) U.S. Cl. CPC C12P 7/16; C12N 1/205; C12N 9/0006; C12N 9/0008; C12R 2001/145; C12R 2001/225; C12R 2001/46; C12Y 101/01157; C12Y 102/01003

(57) ABSTRACT

Described herein are methods to convert a carbohydrate-containing source to a carbonaceous product. In some aspects, the methods include contacting a first portion of the carbohydrate-containing source with a first inoculant comprising a LAB and acetogen, thereby forming a first fermentation mixture; incubating the first fermentation mixture to produce acetate; contacting a second portion of the carbohydrate-containing source with acetate and a second inoculant comprising a solventogenic Clostridia, thereby forming a second fermentation mixture which is incubated to produce the carbonaceous product. Also disclosed herein are fermentation inoculants for the conversion of a carbohydrate-containing source to a carbonaceous product, the inoculant comprising a LAB expressing an enzyme catalyzing the production of lactate from the source, a mixotrophic acetogen expressing an enzyme that catalyzes the production of acetate from lactate and formate, and a solventogenic Clostridia expressing an enzyme that catalyzes the production of the carbonaceous product from the carbohydrate source.

### CROSS REFERENCE TO RELATED APPLICATIONS

This application claims the benefit of priority to U.S. Provisional Application No. 63/401,943, filed Aug. 29, 2022, which is incorporated by reference herein in its entirety.

### STATEMENT ACKNOWLEDGING GOVERNMENT SUPPORT

This invention was made with government support under Grant No. DE-AR0001512 awarded by the Department of Energy. The Government has certain rights in the invention.

### BACKGROUND

Demand for fuel sources, such as butanol, has exponentially increased in recent decades. Butanol is almost exclusively produced via petrochemical routes and used in industrial solvents and in the manufacturing of acrylate esters, amino resins, and butylamines. Although these conventional pathways offer high product yields, they often rely on environmentally deleterious chemicals, making their continued use impractical. More sustainable avenues of generating butanol have begun to emerge as potential alternatives to the conventional approach. For example, microbial fermentation of plant biomass has been explored as one environmentally friendly production method in which renewable feedstocks, such as lignocellulose biomass, can be transformed into usable fuels. However, these fermentation pathways typically suffer from low yields and can result in the release of a suite of greenhouse gases such as carbon dioxide. Thus, there is a need for fermentation methods and inoculants that generate sufficient biofuel yields without the release of damaging byproducts.

### SUMMARY

Described herein are methods related to the conversion of a carbohydrate-containing source to a carbonaceous product. In some aspects, the methods include contacting a first portion of the carbohydrate-containing source with a first inoculant comprising a lactic acid bacterium (LAB) and an acetogen, thereby forming a first fermentation mixture; incubating the first fermentation mixture under conditions effective to produce acetate from carbohydrate, lactate and formate; contacting a second portion of the carbohydrate-containing source with acetate and a second inoculant comprising a solventogenic Clostridia, thereby forming a second fermentation mixture; and incubating the second fermentation mixture under conditions effective to produce the carbonaceous product (e.g., butanol).

Also disclosed herein are methods related to the conversion of a carbohydrate-containing source to acetate. Various aspects of the method include contacting the carbohydrate-containing source with an inoculant comprising a lactic acid bacterium (LAB) and an acetogen, thereby forming a fermentation mixture, wherein the carbohydrate-containing source comprises a carbohydrate, lactate, and formate; and incubating the fermentation mixture under conditions effective to produce acetate from the carbohydrate, lactate and formate.

Additionally described herein are methods for converting a carbohydrate-containing source and acetate to a carbonaceous product. Various aspects of the method contacting the carbohydrate-containing source and acetate with an inoculant comprising a solventogenic Clostridia, thereby forming a fermentation mixture; and incubating the fermentation mixture under conditions effective to produce the carbonaceous product.

In some aspects, the LAB includes a Lactobacillus, a Streptococcus, a Lactococcus, a Pediococcus, or a combination thereof. In various aspects, the LAB includes Lactococcus lactis, Streptococcus lactis, Lactococcus plantarum, Lactobacillus delbrueckii, and Lactobacillus bulgaricus, or a combination thereof.

In some aspects, the acetogen includes Clostridium formicoaceticum, Acetobacterium woodii, Clostridium aceticum, Clostridium ragsdalei, Clostridium thermocellum, Clostridium autoethanogenum, Clostridium ljungdahlii, Moorella thermoacetica, Eubacterium limosum, or a combination thereof.

In some aspects, the solventogenic Clostridia includes Clostridium tyrobutyricum, Clostridium acetobutylicum, Clostridium beijerinkii, Clostridium saccharolyticum, Clostridium carboxidivorans, Clostridium saccharoperbutylacetonicum, Clostridium butylicum, Clostridium cellulovorans, or a combination thereof.

In some aspects, the carbonaceous product includes butanol, butyrate, acetone, isopropanol, ethanol, butanediol, propanediol, butyl butyrate, or a combination thereof. In various aspects, the carbonaceous product includes n-butanol.

In some aspects, the carbohydrate-containing source includes one or more monosaccharides. In some aspects, the one or more monosaccharides include glucose, xylose, or a combination thereof. In some aspects, the carbohydrate-containing source includes a lignocellulose-derived sugar.

In some aspects, the method further includes adding formate to the first fermentation mixture during the first fermentation. In some aspects, the incubation of the second fermentation mixture generates CO₂ and H₂ as byproducts, wherein the method further includes electrochemically reducing the CO₂ to produce recycled formate. In some aspects, the method further includes adding the recycled formate to the first fermentation mixture. In some aspects, the method includes incubating the first fermentation mixture and incubating the second fermentation mixture occur simultaneously.

In some aspects, the first fermentation mixture is incubated in a first bioreactor and the second fermentation mixture is incubated in a second bioreactor.

In some aspects, the first fermentation mixture and the second fermentation mixture are incubated simultaneously in the same bioreactor.

In some aspects, at least one of the lactic acid bacteria, the acetogen, and the solventogenic Clostridia include a genetically modified bacterium.

In some aspects, the method further includes contacting the second fermentation mixture with methyl viologen (MV), benzyl viologen (BV), neutral red, or a combination thereof.

In some aspects, at least one of the lactic acid bacteria, the acetogen, and the solventogenic Clostridia include a genetically modified bacterium. In some aspects, the solventogenic Clostridia can include a Clostridia (e.g., Clostridium tyrobutyricum) engineered to overexpress an aldehyde/alcohol dehydrogenase (adhE2) gene. In some aspects, the solventogenic Clostridia includes a Clostridia (e.g., Clostridium tyrobutyricum) engineered to overexpress an NADPH-dependent beta-hydroxybutyryl-CoA dehydrogenase (hbd) gene. In some aspects, the solventogenic Clostridia can also be a Clostridia (e.g., Clostridium tyrobutyricum) engineered to overexpress an aldehyde: ferredoxin oxidoreductase (aor) gene. In some aspects, the solventogenic Clostridia includes a Clostridia (e.g., Clostridium tyrobutyricum) engineered to overexpress a carboxylic acid reductase (car) gene. In some aspects, the solventogenic Clostridia includes a Clostridia (e.g., Clostridium tyrobutyricum) engineered to overexpress a ferredoxin-NAD(P)⁺ oxidoreductase (fnr or nfnAB) gene. In some aspects, the solventogenic Clostridia includes a Clostridia (e.g., Clostridium tyrobutyricum) engineered to overexpress a formate dehydrogenase (fdh) gene. In some aspects, the solventogenic Clostridia includes an engineered Clostridia (e.g., Clostridium tyrobutyricum) with its CoA transferase (cat1) gene knockout from its genome. In some aspects, the solventogenic Clostridia includes an engineered Clostridia (e.g., Clostridium tyrobutyricum) with its redox-sensing transcriptional repressor gene (rexA) knockout from its genome.

In some aspects, the acetogen includes a carboxydotroph. In some aspects, the method includes incubating the carboxydotroph under conditions effective to convert single carbon compounds in the first fermentation mixture to acetate, and wherein said conversion occurs at 90% or more carbon conversion efficiency.

In some aspects, the LAB includes a homolactic acid bacterium. In some aspects, the method includes incubating the homolactic acid bacteria under conditions effective to convert the first portion of the carbohydrate-containing source to lactate at a yield of 90% or more.

In some aspects, the method further includes collecting a fermentation byproduct from the incubation of the second fermentation mixture, and wherein said fermentation byproduct includes 5% or more of CO₂ by mass.

In some aspects, the acetate produced from the incubation of the first fermentation mixture is used during the incubation of the second fermentation mixture.

Also disclosed herein are fermentation inoculants for the conversion of a carbohydrate-containing source to a carbonaceous product. Various aspects of the fermentation inoculant include a lactic acid bacterium (“LAB”) expressing an enzyme that catalyzes the production of lactate from the carbohydrate-containing source, a mixotrophic acetogen expressing an enzyme that catalyzes the production of acetate from lactate and formate, and a solventogenic Clostridia expressing an enzyme that catalyzes the production of the carbonaceous product from the carbohydrate-containing source and acetate.

In some aspects, at least one of the lactic acid bacteria, the acetogen, and the solventogenic Clostridia include a genetically modified bacterium. In some aspects, the solventogenic Clostridia can include a Clostridia (e.g., Clostridium tyrobutyricum) engineered to overexpress an aldehyde/alcohol dehydrogenase (adhE2) gene. In some aspects, the solventogenic Clostridia includes a Clostridia (e.g., Clostridium tyrobutyricum) engineered to overexpress an NADPH-dependent beta-hydroxybutyryl-CoA dehydrogenase (hbd) gene. In some aspects, the solventogenic Clostridia can also be a Clostridia (e.g., Clostridium tyrobutyricum) engineered to overexpress an aldehyde: ferredoxin oxidoreductase (aor) gene. In some aspects, the solventogenic Clostridia includes a Clostridia (e.g., Clostridium tyrobutyricum) engineered to overexpress a carboxylic acid reductase (car) gene. In some aspects, the solventogenic Clostridia includes a Clostridia (e.g., Clostridium tyrobutyricum) engineered to overexpress a ferredoxin-NAD (P)⁺ oxidoreductase (fnr or nfnAB) gene. In some aspects, the solventogenic Clostridia includes a Clostridia (e.g., Clostridium tyrobutyricum) engineered to overexpress a formate dehydrogenase (fdh) gene. In some aspects, the solventogenic Clostridia includes an engineered Clostridia (e.g., Clostridium tyrobutyricum) with its CoA transferase (cat1) gene knockout from its genome. In some aspects, the solventogenic Clostridia includes an engineered Clostridia (e.g., Clostridium tyrobutyricum) with its redox-sensing transcriptional repressor gene (rexA) gene knockout from its genome. In some aspects, the solventogenic Clostridia includes Clostridium tyrobutyricum.

Also disclosed herein is a system for converting a carbohydrate-containing source to a carbonaceous product. In various aspects, the system includes one or more bioreactors comprising the fermentation inoculant described herein. In some aspects, the one or more bioreactors includes: a first bioreactor; wherein the first bioreactor is configured to receive a first portion of the carbohydrate-containing source, the lactic acid bacterium (“LAB”), and the mixotrophic acetogen, and wherein the first bioreactor is configured to operate under conditions effective to culture the first portion of the carbohydrate-containing source to produce acetate; and a second bioreactor; wherein the second bioreactor is configured to receive a second portion of the carbohydrate-containing source, the acetate, and the solventogenic Clostridia, and wherein the second bioreactor is configured to operate under conditions effective to produce the carbonaceous product.

In some examples, the systems, methods, and compositions described herein can produce a suite of carbonaceous products (e.g., butanol, butyrate, acetone, isopropanol, ethanol, butanediol, propanediol, and/or butyl butyrate) from widely available and renewable sources. Furthermore, the pathways described herein can reduce or eliminate the release of harmful greenhouse gases such as CO₂ typically associated with fermentation reactions while still maintaining high product yields.

### DESCRIPTION OF DRAWINGS

FIG. 1 depicts microbial consortia to produce butanol from glucose and formate (from CO₂/H₂) with lactate and acetate as intermediary metabolites. The LAB (e.g., Lactococcus lactis) converts glucose to lactate and removes trace oxygen dissolved in the culture medium and, thereby, establishes a conducible environment for strict anaerobes in the consortium. The mixotrophic acetogen (e.g., Clostridium formicoaceticum), which cannot use glucose, converts lactate and formate to acetate. A solventogenic Clostridia (e.g., Clostridium tyrobutyricum) converts glucose and acetate to butyrate and butanol as the final product via two pathways involving aldehyde/alcohol dehydrogenases (ALD, ADH) and ferredoxin-associated aldehyde oxidoreductase (AOR). CO₂ from sugar fermentation (and externally supplied) is converted via electrochemical reduction to formic acid that is used by the mixotrophic acetogen to produce acetate.

FIG. 2 depicts butanol production from lignocellulose hydrolysate sugars (glucose and xylose) in fermentation with LAB, mixotrophic acctogen, and butanol-producing Clostridia. CO₂ is electrochemically reduced to formate. For illustration, the mixotrophic and Clostridial fermentations are shown in two steps, but the inoculants can be housed together or in separate bioreactors.

FIG. 3 illustrates C. tyrobutyricum with engineered pathways for butanol biosynthesis from sugars and acetate. The wild-type strain produces butyrate and acetate as two main metabolites. The heterologous gene adhE2 encoding a bifunctional aldehyde/alcohol dehydrogenase enables the biosynthesis of butanol and ethanol from butyryl-CoA and acetyl-CoA, respectively. Additional reducing equivalents (NADH and NADPH) for butanol biosynthesis can be provided with expressing fdh encoding formate dehydrogenase, fnr and nfnAB encoding ferredoxin-NAD (P)+ oxidoreductase, and hbd encoding a NADP+-dependent 3-hydroxybutyryl-CoA dehydrogenase. Adding an artificial electron carrier such as methyl viologen, which inhibits hydrogen production, also increases NADH availability for butanol biosynthesis. Acetate is assimilated through the butyryl-CoA: acetate CoA transferase encoded by cat1, which is also responsible for the biosynthesis of butyrate. Butyrate can be reduced to butyraldehyde by aldehyde: ferredoxin oxidoreductase encoded by aor and NADPH-dependent carboxylic acid reductase encoded by car.

FIGS. 4A-4C depict fermentation kinetics of C. formicoaceticum grown on various carbon sources. FIG. 4A illustrates cell growth monitored as OD₆₀₀; FIG. 4B illustrates acetate production from various carbon sources; FIG. 4C illustrates acetate production from lactate and formate.

FIGS. 5A-5B show acetate production from lactose (FIG. 5A) and glucose (FIG. 5B) in a cocultured fermentation with L. lactis and C. formicoaceticum, which formed a consortium with intimate contacts co-immobilized in a fibrous matrix (SEM picture).

FIG. 6A-6F depict batch fermentation kinetics of glucose by C. tyrobutyricum overexpressing adhE2 and hbd. WT-adhE2 (FIG. 6A); WT-adhE2-hbd (FIG. 6B); Δack-adhE2 (FIG. 6C), Δack-adhE2-hbd (FIG. 6D); Δcat1::adhE2 (FIG. 6E), Δcat1::adhE2-hbd (FIG. 6F). The fermentations were carried out in serum bottles with CaCO₃ to buffer the pH at ˜5.5 and at ˜37° C.

FIGS. 7A-7B show comparisons of butanol yields and C4/C2, alcohol/acid, and butanol/ethanol ratios from different strains WT-adhE2, Δack-adhE2, and Δcat1::adhE2 (FIG. 7A) and those also overexpressing hbd (FIG. 7B) in batch fermentations.

FIGS. 8A-8F are plots showing batch fermentation kinetics of glucose by C. tyrobutyricum Δack-adhE2-hbd and WT-adhE2-hbd in biorcactors with pH controlled at 6.5 (FIGS. 8A and 8D), 6.0 (FIGS. 8B and 8E), and 5.5 (FIGS. 8C and 8F), respectively with Ca(OH)₂ and at ˜37° C.

FIGS. 9A-9B show comparisons of specific growth rates (μ), butanol yields, and C4/C2, alcohol/acid, and butanol/ethanol ratios at different pHs in batch fermentations with Δack-adhE2-hbd (FIG. 9A) and WT-adhE2-hbd (FIG. 9B).

FIGS. 10A-10F depict batch fermentation kinetics of glucose by C. tyrobutyricum Δcat1: adhE2 in serum bottles in the presence of methyl viologen (MV) at various concentrations (FIGS. 10A-10E) and a comparison of the effects of MV on butanol yield (FIG. 10F), and C4/C2, alcohol/acid, and butanol/ethanol ratios (FIG. 10F). The batch fermentation kinetics include MV at concentrations of 0 μM (FIG. 10A); 100 μM (FIG. 10B); 200 μM (FIG. 10C); 500 μM (FIG. 10D); and 1000 μM (FIG. 10E).

FIGS. 11A-11H depict batch fermentation kinetics of glucose (FIG. 11A-11D) and xylose (FIGS. 11E-11H) by C. tyrobutyricum Δcat1::adhE2 in bioreactors in the presence of methyl viologen (MV) at various concentrations (0 mM, 200 mM, 500 mM, 1000 mM). For glucose fermentation, MV was added at 12 h after inoculation. For xylose fermentation, MV addition was delayed to 24 h for the higher concentrations of 500 mM and 1000 mM, which completely inhibited cell growth and fermentation.

FIG. 12 shows the effects of MV on fermentation kinetics of C. tyrobutyricum Δcat1::adhE2 grown on glucose (Left) and xylose (Right), respectively, in bioreactors. The fermentation time-course data are shown in FIGS. 11A-11H. Xylose gave higher specific growth rate and butanol production at the lower MV concentrations.

FIGS. 13A-13E depict batch fermentation kinetics of C. tyrobutyricum Sack-adhE2-hbd with glucose and lactate as co-substrates at different lactate concentrations from 0 to ˜26 g/L in serum bottles.

FIG. 14 depicts fermentation kinetics of C. tyrobutyricum Ct-pTBA with glucose/xylose mixture (1:1) as substrates and benzyl viologen (BV) as an artificial electron carrier at pH 6.0. 18 g/L acetate added at ˜48 h was all re-assimilated to produce additional butyrate in the fermentation.

FIG. 15 shows repeated-batch fermentation by C. tyrobutyricum Δack-adhE2 with hydrolysate sugars from various lignocelluloses-cotton stalk (CSH), sugarcane bagasse (SBH), soybean hull (SHH), and corn fiber (CFH)-in the presence of 250 μM methyl viologen (MV250) or 25 μM benzyl viologen (BV25). Xylose was used in the first batch as a control whereas CSH, SBH, SHH, or CFH with MV250 or BV25 were used in subsequent batches. High butanol yield of ˜0.3 g/g and productivity of up to 0.35 g/L·h was obtained in CSH fermentations. Butanol yields from SBH, SHH, and CFH were lower at 0.22, 0.27, and 0.17 g/g, respectively.

FIG. 16 shows butanol production from glucose by Ct-adhE2-aor2 (Cf) and Ct-adhE2-aor (Cc) in batch fermentations at pH 5.5, 6.0, and 6.5. About 200 μM methyl viologen (MV) was added at 12 h.

FIG. 17 depicts an anaerobic fermentation pathways for biosynthesis of various carboxylic acids and solvents from organic carbon feedstocks.

FIG. 18 depicts a schematic diagram illustrating an example system and components for the conversion of a carbohydrate-containing source into carbonaceous products.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### General Definitions

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise.

As used herein, the terms “optional” or “optionally” mean that the subsequently described event or circumstance may or may not occur and that the description includes instances where said event or circumstance occurs and instances where it does not.

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims, which follow, reference will be made to a number of terms that shall be defined herein.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 10% (e.g., within 5%, 2%, or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the term “substantially” means that the subsequently described event or circumstance completely occurs or that the subsequently described event or circumstance generally, typically, or approximately occurs. As used herein, the term “substantially,” in, for example, the context “substantially identical” or “substantially similar” refers to a method or a system, or a component that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the term “conditions effective” as it relates to the inoculation of a fermentation mixture refers to a set of adjustable process parameters, such as pH, temperature, metabolite environment, and time, which can produce a desired product. For example, the incubation of a fermentation mixture under conditions effective can convert a compound or compounds, such as single carbon compounds, to a target product, such as acetate, at an efficiency.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

The present invention may be understood more readily by reference to the following detailed description of various aspects of the invention and the examples included therein and to the Figures and their previous and following description.

### Methods

Disclosed herein are methods related to the conversion of a carbohydrate-containing source to a carbonaceous product. Various aspects of the method include contacting a first portion of the carbohydrate-containing source with a first inoculant comprising a lactic acid bacterium (LAB) and an acetogen, thereby forming a first fermentation mixture; incubating the first fermentation mixture under conditions effective to produce acetate from carbohydrate, lactate and formate; contacting a second portion of the carbohydrate-containing source with the acetate and a second inoculant comprising a solventogenic Clostridia, thereby forming a second fermentation mixture; and incubating the second fermentation mixture under conditions effective to produce the carbonaceous product. The carbon dioxide released from the second fermentation mixture can be captured and converted to formate that is fed to the first fermentation mixture for acetate production. In various aspects, the first fermentation mixture and second fermentation mixture are contained within a single bioreactor.

Also disclosed herein are methods related to the conversion of a carbohydrate-containing source to acetate. Various aspects of the method include contacting the carbohydrate-containing source with an inoculant comprising a lactic acid bacterium (LAB) and an acetogen, thereby forming a fermentation mixture, wherein the carbohydrate-containing source comprises a carbohydrate, lactate, and formate; and incubating the fermentation mixture under conditions effective to produce acetate from the carbohydrate, lactate and formate.

Additionally described herein are methods for converting a carbohydrate-containing source and acetate to a carbonaceous product. Various aspects of the method contacting the carbohydrate-containing source and acetate with an inoculant comprising a solventogenic Clostridia, thereby forming a fermentation mixture; and incubating the fermentation mixture under conditions effective to produce the carbonaceous product.

As used herein, “lactic acid bacteria” and “LAB” refer to a group of Gram-positive, acid-tolerant, generally non-sporulating, non-respiring bacteria that produce lactic acid as the metabolic end product of carbohydrate fermentation. The bacteria are often found in decomposing plants and lactic products. Illustrative bacterial genera include, but are not limited to, Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, Atopobium, and Weisella. Illustrative sources of lactic acid bacteria isolates include, but are not limited to, the intestine of species such as Gibelion catla, Nibea sp., Labeo rohita, Raiamas bola, Puntius sp., Oreochromis niloticus, Heteropneutes fossilis, Sus scrofa domesticus, Gallus domesticus, or Capra aegagrus hircus. In some aspects, the LAB includes a Lactobacillus, a Streptococcus, a Lactococcus, or a Pediococcus. For example, the LAB of the present method can include from one or more of Lactococcus lactis, Streptococcus lactis, Lactococcus plantarum, Lactobacillus delbrueckii, and Lactobacillus bulgaricus. In some aspects, the LAB includes a homolactic acid bacterium and incubation of the homolactic acid bacteria under conditions effective can convert the first portion of the carbohydrate-containing source to lactate at a yield of 90% or more, such as 95% or more, 97% or more, or 99% or more.

As used herein, “acetogen” or “acetogenic organism” refers to a microorganism that generates acetate as a product of autotrophic or heterotrophic anaerobic respiration. The term “homoacetogen” refers to a strictly anaerobic microorganism that catalyzes the formation of acetate as the sole or major metabolic product from organic carbon substrates such as sugar and lactate and C₁ compounds including formate, most of which are able to grow at the expense of hydrogen and/or CO as the sole energy source. The terms “carboxydotroph” and “carboxydotrophic organism” refer to an organism that can tolerate high concentrations of carbon monoxide, and generally are capable of utilizing carbon monoxide for metabolism. A carboxydotrophic microorganism can furthermore obtain energy and carbon from the oxidation of CO. Such a carboxydotrophic microorganism can be an aerobic bacterium capable of oxidizing CO to carbon dioxide. A carboxydotrophic microorganism can also be an obligate anaerobe, which can be capable of reducing carbon dioxide to CO and/or converting CO to carbon dioxide and hydrogen. Some carboxydotrophic microorganisms that are an obligate anaerobe are acetogenic bacteria and can form acetate from CO and/or CO₂. In some aspects, the acetogen includes one or more of Clostridium formicoaceticum, Acetobacterium woodii, Clostridium aceticum, Clostridium ragsdalei, Clostridium thermocellum, Clostridium autoethanogenum, Clostridium ljungdahlii, Moorella thermoacetica, or Eubacterium limosum. In some examples, the acetogen includes a carboxydotroph and the method includes incubating the carboxydotroph under conditions effective to convert single carbon compounds in the first fermentation mixture to acetate, and wherein said conversion occurs at 90% or more carbon conversion efficiency, such as at 95% or more carbon conversion efficiency, 97% carbon conversion efficiency, or 99% carbon conversion efficiency.

As used herein, the term “solventogenic Clostridia” can refer to anaerobic, endospore forming bacteria that produce a large array of primary metabolites, such as butanol and butyric acid, by anaerobically degrading simple and complex carbohydrates, including starch, cellulose and hemicellulose. Clostridium tyrobutyricum, Clostridium acetobutylicum, Clostridium beijerinkii, Clostridium saccharolyticum, Clostridium carboxidivorans, Clostridium saccharoperbutylacetonicum, Clostridium butylicum, and Clostridium cellulovorans are some suitable examples of solventogenic Clostridia.

Various carbohydrate-containing sources can be converted into carbonaceous products or acetate using these methods, such as types of monosaccharides, oligosaccharides, and/or polysaccharides. In various examples, the carbohydrate-containing source includes a lignocellulose hydrolysate. As used herein, the term “lignocellulose hydrolysate” refers to hydrolysis products of lignocellulose or lignocellulosic material comprising cellulose and/or hemicellulose, oligosaccharides, mono- and/or disaccharides, acetic acid, formic acid, other organic acids, furfural, hydroxymethyl furfural, levulinic acid, phenolic compounds, other hydrolysis and/or degradation products formed from lignin, cellulose, hemicellulose and/or other components of lignocellulose, nitrogen compounds originating from proteins, metals and/or non-hydrolyzed or partly hydrolyzed fragments of lignocellulose. In some examples, lignocellulose hydrolysates are obtained from a lignocellulosic biomass such as paper, paper products, wood, wood-related materials, particle board, grasses, rice hulls, bagasse, cotton, jute, hemp, flax, bamboo, sisal, abaca, straw, corn cobs, coconut hair, algae, seaweed, altered celluloses, e.g., cellulose acetate, regenerated cellulose, and the like, or combinations thereof. The lignocellulosic biomass may be pretreated (e.g., using physical, chemical, or biological processes such as steam explosion, acid or alkaline treatments, ammonia fiber expansion, or enzymatic pretreatment) prior to carrying out hydrolysis and any subsequent separation/purification step. In various aspects, the carbohydrate-containing source includes a sugar derived from lignocellulose. In some aspects, the carbohydrate-containing source includes one or more of a starch, sucrose, lactose, fructose, galactose, and pentoses (such as xylose). In some examples, the carbohydrate-containing source includes a monosaccharide such as glucose, xylose, or a combination thereof.

The methods can further include adding formate to the first fermentation mixture during the first fermentation. In some aspects, the incubation of the second fermentation mixture generates CO₂ and H₂. In some examples, the CO₂ is electrochemically reduced to produce recycled formate. In electrochemical reduction, electrons are transferred to CO₂ at the cathode and electrolysis of water releases electrons to the anode. The overall reaction (CO₂+H₂O→HCOOH+0.5 O₂) also releases 0.5 O₂. Thus, formate can be generated from the byproduct produced in the fermentation process using only electricity and water. Cathodic materials comprising materials suitable for large-scale industrial operation working at different conditions (pH, current density, temperature), such as palladium (Pd) and carbon nanotubes (CNT), can achieve a formate faradaic efficiency of >80%. In various aspects, the reduction of the CO₂ and H₂ byproducts is done without the use of catalysts. However, in some aspects, the resulting CO₂ is electrocatalytically reduced using direct CO₂ hydrogenation. This recycled formate can, in some examples, be supplied back to the first fermentation mixture, thereby promoting mixotrophic fermentation of the acetogen. In some examples, the recycled formate is supplied back to the second fermentation mixture. In various aspects, the rate at which the recycled formate is added back to the fermentation mixtures can be controlled such that the amount of formate in the first fermentation mixture is maintained within a non-toxic level to the microorganisms in the first inoculant. This can be achieved by feeding the recycled formate to the first fermentation mixture at a rate sufficient for simultaneous consumption by the first inoculant. In some examples, the recycled formate is introduced to the first fermentation mixture by intermittently injecting an amount of the formate at discrete intervals to reach a desired concentration within the mixture. In some other examples, the recycled formate is continuously added to the first fermentation mixture.

The present methods can additionally include adding certain hydrogenase inhibitors and/or electron carriers to either the first and/or second fermentation mixture. Hydrogenase inhibitors include substances or compounds that can be used to selectively inhibit the activity of hydrogenase enzymes, thereby decreasing the resulting production of hydrogen. In some aspects, artificial electron carriers can be added to inhibit hydrogen production, while also increasing NADH availability for butanol biosynthesis. For example, in some aspects, the method includes contacting the second fermentation mixture with methyl viologen (MV), benzyl viologen (BV), neutral red, or a combination thereof. Reducing hydrogen production by blocking or inhibiting hydrogenase can direct more reducing power to butanol production, and lead to greater butanol yields.

Various aspects of the present methods include simultaneously incubating the first fermentation mixture and the second fermentation mixture. In some aspects, the first fermentation mixture is incubated in a first bioreactor and the second fermentation mixture is incubated in a second bioreactor. In other examples, the first fermentation mixture and the second fermentation mixture are incubated simultaneously in the same bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some aspects, one or more of the bioreactors can include a growth reactor which can be used to seed a fermentation reactor. Bioreactors may range in size from a few liters to several cubic meters (i.e., several 1000 liters) or larger and can be formed using a number of different materials (e.g., stainless steel or glass).

Based on the mode of operation, a bioreactor may be classified as batch, fed-batch or continuous. The bioreactor is typically equipped with one or more inlets for supplying culture medium to the inoculants, and with one or more outlets for harvesting product or emptying the bioreactor. Additionally, the bioreactor may be equipped with at least one outlet constructed in such a way that a separation device can be attached to the bioreactor. Typically, the bioreactor's environmental conditions such as gas (i.e., air, oxygen, nitrogen, carbon dioxide) flow rates, temperature, pH and dissolved oxygen levels, and agitation speed/circulation rate can be closely monitored and controlled. In various aspects, the fermentation mixture(s) are maintained in an aqueous culture medium including nutrients, vitamins, and/or minerals sufficient to permit growth of the microorganism(s). Suitable media are well known in the art as instructed by this disclosure.

The present methods can use a set of fermentation reactions to convert the carbohydrate-containing source or acetate to a carbonaceous product, such as a biofuel. As used herein, the term “carbonaceous product” refers to materials that contain of substantial amounts of carbon. In some aspects, the carbonaceous product includes one or more of butanol, butyrate, acetone, isopropanol, ethanol, butanediol, propanediol, or butyl butyrate. In some examples, the carbonaceous product formed by the incubation of the second fermentation mixture is n-butanol. In some aspects, the acetate produced from the incubation of the first fermentation mixture is used during the incubation of the second fermentation mixture.

In some aspects, at least one of the lactic acid bacteria, the acetogen, and the solventogenic Clostridia include a genetically modified bacterium. Some examples of methods for engineering Clostridium can be found in U.S. Pat. Nos. 8,450,093 and 9,284,580. For example, the solventogenic Clostridia can include a Clostridia (e.g., Clostridium tyrobutyricum) engineered to overexpress an aldehyde/alcohol dehydrogenase (adhE2) gene. In some examples, the solventogenic Clostridia includes a Clostridia (e.g., Clostridium tyrobutyricum) engineered to overexpress an NADPH-dependent beta-hydroxybutyryl-CoA dehydrogenase (hbd) gene. The solventogenic Clostridia can also include a Clostridia (e.g., Clostridium tyrobutyricum) engineered to overexpress an aldehyde: ferredoxin oxidoreductase (aor) gene. In some aspects, the solventogenic Clostridia includes a Clostridia (e.g., Clostridium tyrobutyricum) engineered to overexpress a carboxylic acid reductase (car) gene. In some aspects, the solventogenic Clostridia comprises a Clostridia (e.g., Clostridium tyrobutyricum) engineered to overexpress a ferredoxin-NAD (P)⁺ oxidoreductase (fnr or nfnAB) gene. The solventogenic Clostridia can also be a Clostridia (e.g., Clostridium tyrobutyricum) engineered to overexpress a formate dehydrogenase (fdh) gene. In some aspects, the solventogenic Clostridia comprises an engineered Clostridia (e.g., Clostridium tyrobutyricum) with its CoA transferase (cat1) gene knockout from its genome. In some aspects, the solventogenic Clostridia comprises an engineered Clostridia (e.g., Clostridium tyrobutyricum) with its redox-sensing transcriptional repressor gene (rexA) gene knockout from its genome. In some examples, the solventogenic Clostridia comprises an engineered Clostridia (e.g., Clostridium tyrobutyricum) with its In various aspects, the inoculant includes engineered Clostridia having a plurality of genetic modifications (e.g., 2 or more genetic modifications, 3 or more genetic modifications, 4 or more genetic modifications, 5 or more genetic modifications).

In various aspects, the method further includes collecting a fermentation byproduct from the incubation of the second fermentation mixture. For example, the fermentation byproduct can include 5% or more of CO₂ by mass, such as 10% or more, 15% or more, 20% or more, 25% or more, 30% or more, 35% or more, 40% or more, 45% or more, 50% or more, or 55% or more of CO₂ by mass. For example, the fermentation byproduct can include 80% or less of CO₂ by mass, such as 75% or less, 70% or less, 65% or less, 60% or less, 55% or less, 50% or less, 45% or less, 40% or less, 35% or less, 30% or less, 25% or less, 20% or less, 15% or less, or 10% or less of CO₂ by mass. In some examples, the amount of CO₂ by mass can be in a range between any of the lower values to any of the upper values.

### Fermentation Inoculant

Also disclosed herein are fermentation inoculants for the conversion of a carbohydrate-containing source to a carbonaceous product or acetate. The term “fermentation inoculant” as used herein refers to a collection of microbial species, or strains of a species, that can be described as carrying out a particular function, or can be described as participating in, or leading to, or correlating with, a recognizable parameter or phenotypic trait. The individual microbes microbial species, or strains of a species of the inoculant may be collocated within a single vessel/container or separated into discrete vessels/containers. Various aspects of the fermentation inoculant include a lactic acid bacterium (“LAB”) expressing an enzyme that catalyzes the production of lactate from the carbohydrate-containing source, a mixotrophic acetogen expressing an enzyme that catalyzes the production of acetate from lactate and formate, and a solventogenic Clostridia expressing an enzyme that catalyzes the production of the carbonaceous product from the carbohydrate-containing source and acetate.

In some aspects, at least one of the lactic acid bacteria, the acetogen, and the solventogenic Clostridia include a genetically modified bacterium. As described herein, “engineered” or “modified” organisms are produced via the introduction of genetic material into a host or parental microorganism of choice thereby modifying or altering the cellular physiology and biochemistry of the microorganism. Through the introduction of genetic material the parental microorganism can acquire new properties, e.g., the ability to produce a new, or greater quantities of, an intracellular metabolite. In an illustrative aspect, the introduction of genetic material into a parental microorganism acquires new properties, e.g., the ability to produce a new, or greater quantities of, an intracellular metabolite. In an illustrative aspect, the introduction of genetic material into a parental microorganism results in a new or modified ability to produce n-butanol. The genetic material introduced into the parental microorganism contains gene(s), or parts of genes, coding for one or more of the enzymes involved in a biosynthetic pathway for the production of n-butanol and may also include additional elements for the expression and/or regulation of expression of these genes, e.g. promoter sequences.

An engineered or modified microorganism can also include in the alternative or in addition to the introduction of a genetic material into a host or parental microorganism, the disruption, deletion, or knocking out of a gene or polynucleotide to alter the cellular physiology and biochemistry of the microorganism. Through the reduction, disruption or knocking out of a gene or polynucleotide the microorganism acquires new or improved properties (e.g., the ability to produce a new or greater quantities of an intracellular metabolite, improve the flux of a metabolite down a desired pathway, and/or reduce the production of undesirable by-products). For example, the microorganism may be modified to express one or more exogenous genes if these genes are introduced into the microorganism with all the elements allowing their expression in the host microorganism. A microorganism may also be modified to modulate the expression level of an endogenous gene. In particular, a genetic modification of a microorganism may be carried out by using techniques known in the art, such as CRISPR-Cas systems described in U.S. Pat. No. 11,142,751.

Some examples of engineered Clostridia can be found in U.S. Pat. Nos. 8,450,093 and 9,284,580. For example, the solventogenic Clostridia can include a Clostridia (e.g., Clostridium tyrobutyricum) engineered to overexpress an aldehyde/alcohol dehydrogenase (adhE2) gene. In some cases, the solventogenic Clostridia comprises a Clostridia (e.g., Clostridium tyrobutyricum) engineered to overexpress an NADPH-dependent beta-hydroxybutyryl-CoA dehydrogenase (hbd) gene. The solventogenic Clostridia can also be a Clostridia (e.g., Clostridium tyrobutyricum) engineered to overexpress an aldehyde: ferredoxin oxidoreductase (aor) gene. In some aspects, the solventogenic Clostridia comprises a Clostridia (e.g., Clostridium tyrobutyricum) engineered to overexpress a carboxylic acid reductase (car) gene. In some aspects, the solventogenic Clostridia comprises a Clostridia (e.g., Clostridium tyrobutyricum) engineered to overexpress a ferredoxin-NAD (P)⁺ oxidoreductase (fnr or nfnAB) gene. The solventogenic Clostridia can also be a Clostridia (e.g., Clostridium tyrobutyricum) engineered to overexpress a formate dehydrogenase (fdh) gene. In some aspects, the solventogenic Clostridia comprises an engineered Clostridia (e.g., Clostridium tyrobutyricum) with its CoA transferase (cat1) gene knockout from its genome. In some aspects, the solventogenic Clostridia comprises an engineered Clostridia (e.g., Clostridium tyrobutyricum) with its redox-sensing transcriptional repressor gene (rexA) gene knockout from its genome. In various aspects, the inoculant includes engineered Clostridia having a plurality of genetic modifications (e.g., 2 or more genetic modifications, 3 or more genetic modifications, 4 or more genetic modifications, 5 or more genetic modifications). In various aspects, the inoculant includes engineered Clostridia having a combination of the above genetic modifications.

### Systems

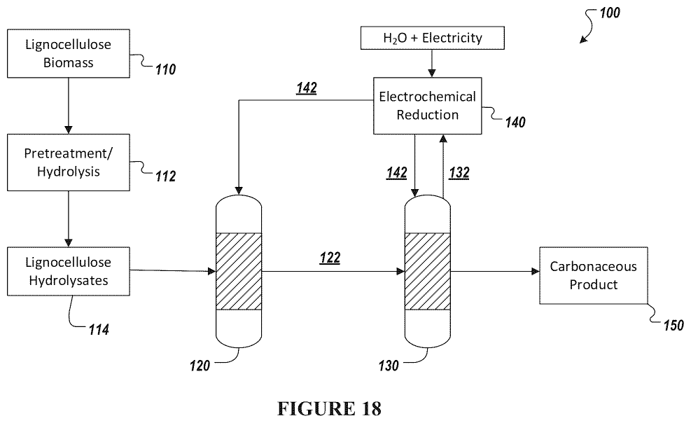

FIG. 18 shows a schematic diagram of an example system 100 for converting a carbohydrate-containing source to a carbonaceous product 150. Raw lignocellulose biomass 110 is exposed to pretreatment and hydrolysis 112 to produce a carbohydrate-containing source (shown as lignocellulose hydrolysate 114). The lignocellulose hydrolysate 114 is fed to a first bioreactor 120 of the system 100. The first bioreactor 120 is configured to receive a first portion of the lignocellulose hydrolysates 114, a lactic acid bacterium (“LAB”), and a mixotrophic acetogen. The lactic acid bacterium (“LAB”) and mixotrophic acetogen can include any of the wild-type or genetically modified bacteria as described above. The first bioreactor 120 is further configured to operate under conditions effective to culture the first portion of the lignocellulose hydrolysate 114 in the presence of lactate and formate to produce acetate.

System 100 further includes a second bioreactor 130 configured to receive a fermentation product 122 from the first bioreactor 120 and a solventogenic Clostridia. The solventogenic Clostridia can include any of the wild-type or genetically modified bacteria as described above. The fermentation product 122 includes a second portion of the lignocellulose hydrolysates 114 and acetate. The second bioreactor is configured to operate under conditions effective to produce the carbonaceous product 150 and a gaseous stream 132 containing CO₂ and H₂. The gaseous stream 132 can be electrochemically reduced 140 with water and electricity to recycle formate 142 to the first bioreactor 120 and second bioreactor 130. Although the system 100 shown in FIG. 18 depicts two bioreactors, other systems can instead use a single bioreactor or more than two bioreactors (e.g., 3 bioreactors, 4 bioreactors, 5 bioreactors, 6 bioreactors, 7 bioreactors, 8 bioreactors, 9 bioreactors, or 10 bioreactors). As described above, bioreactors typically include a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some aspects, one or more of the bioreactors can include a growth reactor which can be used to seed a fermentation reactor. Bioreactors can range in size from a few liters to several cubic meters (i.e. several 1000 liters) or larger and can be formed using a number of different materials e.g. stainless steel or glass.

Based on the mode of operation, a bioreactor may be classified as batch, fed-batch or continuous. The bioreactor is typically equipped with one or more inlets for supplying culture medium to the cells, and with one or more outlets for harvesting product or emptying the bioreactor. Additionally, the bioreactor may be equipped with at least one outlet constructed in such a way that a separation device can be attached to the bioreactor. Typically, the bioreactor's environmental conditions such as gas (i.e., air, oxygen, nitrogen, carbon dioxide) flow rates, temperature, pH and dissolved oxygen levels, and agitation speed/circulation rate can be closely monitored and controlled. In various aspects, the fermentation mixture(s) are maintained in an aqueous culture medium including nutrients, vitamins, and/or minerals sufficient to permit growth of the microorganism(s). Suitable media are well known in the art as instructed by this disclosure.

### Examples

The following examples are put forth so as to provide those of ordinary skill in the art with a complete disclosure and description of how the compounds, compositions, articles, devices, and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by volume, the temperature is in degrees C. or is at ambient temperature, and pressure is at or near atmospheric.

### INTRODUCTION

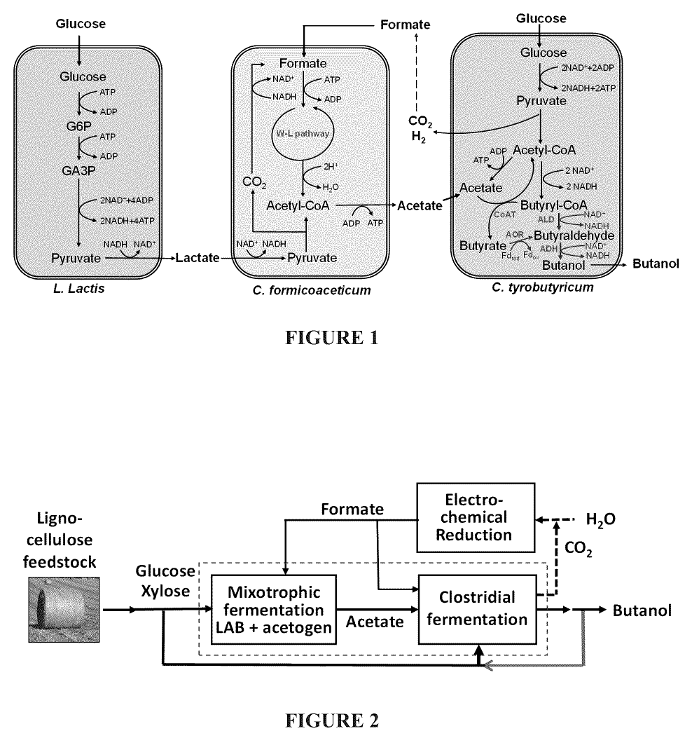

Engineered mixotrophic consortia with external reducing equivalents described hereafter can avoid/minimize CO₂ evolution and optimize the carbon efficiency. The microbial consortia can be used for carbon optimized bioconversion in an integrated bioprocess that facilitates multi-trophic carbon flux and utilization. Such a defined microbial consortia can utilize, recapture and/or recycle gaseous CO₂ into products such as butanol and butyric acid (FIG. 1). The microbial consortia can also be used to produce butyrate and butanol in an integrated fermentation process with formate produced from electrochemical reduction of CO₂ and glucose and xylose from lignocellulose hydrolysates (FIG. 2). A yield of ˜0.56 g/g, butanol can be produced at a cost of less than $2.5/gal with a >50% reduction in greenhouse gas (GHG) emissions.

Converting CO₂ released in the sugar fermentation to formate allows its efficient conversion to acetate by the acetogen under mixotrophic conditions with lactate as a co-substrate. Among other things, this design overcomes the difficulty in delivering external reducing equivalents required for CO₂ fixation by carboxydotrophic bacteria via the Wood-Ljungdahl (W-J) pathway, also known as the reductive acetyl-CoA pathway. Compared to CO and H₂, formate with a high solubility in water and −420 mV redox potential is a better external energy carrier and can provide the needed reducing equivalents for carbon conversion and alcohol biosynthesis. Formate can similarly be regenerated electrochemically or produced electrocatalytically from CO₂, H₂ and H₂O. Thus, the reducing equivalent generation system is decoupled from its delivery to the proposed bioconversion system, which can accommodate external reducing equivalents to optimize the carbon efficiency and avoid CO₂ evolution.

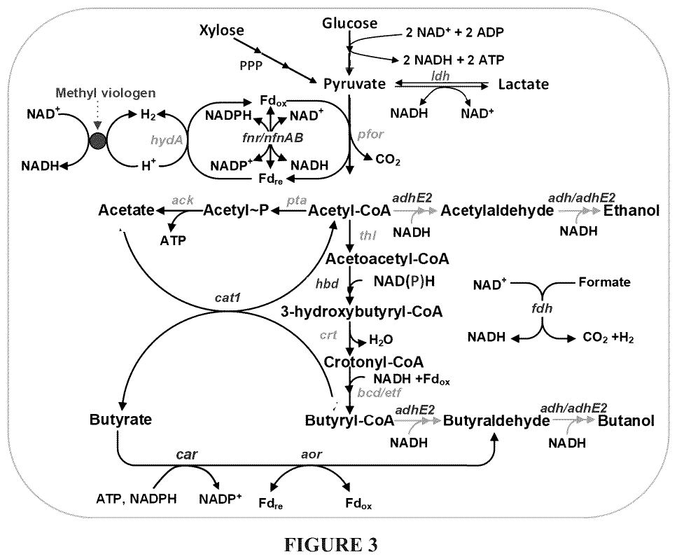

Some Clostridia, such as C. tyrobutyricum, can assimilate acetate, in the presence of glucose or xylose, to butyrate through CoA transferase (CoAT). Engineered C. tyrobutyricum mutant strain overexpressing adhE2, encoding aldehyde dehydrogenase (ALD) and alcohol dehydrogenase (ADH) can produce the highest butanol titer, yield, and productivity from glucose and xylose among all known butanol-producing microbes including engineered E. coli and solventogenic clostridia. FIG. 3 illustrates the metabolic pathways in engineered C. tyrobutyricum for butanol production from sugars and acetate. A more robust strain that also expresses aor, encoding aldehyde: ferredoxin oxidoreductase (AOR), can provide an effective route to convert butyrate to butanol. AOR plays an important role in ATP generation under energy-limited conditions in acetogens, but its potential role in alcohol production is not fully understood. The AOR in C. formicoaceticum (and other acetogens) has been shown to convert a wide range of carboxylates to corresponding alcohols in the presence of CO or formate and an electron mediator such as the reduced methyl viologen (MV⁻). AOR from Aromatoleum aromaticum catalyzes the reduction of organic acids with low-potential Eu(II) or Ti(III) complexes as well as with H₂ as an electron donor. Therefore, the reduction of butyrate to butyraldehyde through AOR can provide an energy-efficient way for butanol production. In addition, butyrate can also be reduced to butyraldehyde through NADPH-dependent carboxylic acid reductase (CAR). With increased butanol production from sugars in engineered solventogenic Clostridia and recapturing CO₂/H₂ for further conversion via formate to acetate, then butyrate, and finally butanol, the overall butanol yield from glucose can reach ˜0.56 g/g, which is over 100% more than that (˜0.25 g/g) obtained in conventional acetone-butanol-ethanol (ABE) fermentation and ˜25% more than ethanol produced from sugar (˜0.45 g/g) in conventional yeast fermentation.

Moreover, the synthetic microbial consortia can create unique niches for the growth of otherwise difficult or unculturable bacteria, such as those highly sensitive to oxygen and metabolic inhibitors. The growth of facultative anaerobic lactic acid bacteria (LAB) will remove trace oxygen dissolved in the culture medium and, thereby, establish a culture environment suitable for strict anaerobes such as acetogens and most clostridia to grow in industrial fermentation. The synthetic microbial consortia with balanced interactions among the component species via population abundance control can optimize the carbon conversion and maximize carbon and energy efficiency with zero CO₂ emission.

The synthetical microbial consortia can substantially reduce CO₂ emissions and increase butanol yield from lignocellulose sugars by 50% (0.618 g/g vs. 0.412 g/g). Several reactions involved in the consortia system and their respective carbon conversion rate and theoretical yield are listed below in Table 1.

TABLE 1
Theoretical yields and conversion rates
of the microbial consortia system.

| | C | |
|---|---|---|
| | conversion | Yield |
| Reactions | (%) | (g/g) |
| LAB: Glucose → 2 Lactate | 100 | 1.0 |
| Mixotrophic acetogen: Lactate + Formate → 2 Acetate | 100 | 1.0 |
| CtΔackΔcat1::adhE2: Glucose → Butanol + 2 | 66.67 | 0.412 |
| CO₂ + 2 H₂ | | |
| CtΔack::aor-adhE2: 2 Acetate → Butyrate → Butanol | 100 | 0.618 |
| Electrochemical: CO₂ + H₂ + 2 e⁻ → Formate | 100 | 1.0 |
| Overall: Glucose + 2 e⁻ → 1.5 Butanol | 100 | 0.618 |

Detailed reaction chemistry and mass balance are given in Table 2. Considering some substrate carbon would be used for cell biomass in fermentation, a possible butanol yield of ˜0.56 g/g glucose for a ˜90% overall carbon conversion efficiency can be achieved. Thus, the system can have a specific energy ratio U_{Butanol}/U_{Glucose} of 1.435. The consortia in a high-cell-density (HCD) fermentation (with either cell recycle or immobilization) can achieve an overall butanol production rate of greater than 2 g/L·h or 72 kJ_{Butanol}/L·h. The mixotrophic fermentation itself can have an acetate production rate of >3 g/L·h or 43.8 kJ_{Acetate}/L·h. This is a highly beneficial compared to conventional gas fermentation process, which usually suffers from a low productivity of <0.1 g/L·h due to low solubility and poor uptake of the gas substrates (CO₂, H₂, CO). In addition, no carboxydotrophic nor mixotrophic acetogen can produce butanol from syngas or CO₂/H₂ at a meaningful titer, yield, or productivity. The synthetic consortia thus can achieve >1 for U_{Butanol}/U_{Glucose} and avoid CO₂ evolution by ˜100%. Low-cost sugars such as glucose and xylose present in the lignocellulose hydrolysates can be used in industrial fermentation process for butanol production. With a 50% higher yield and much higher productivity, butanol can be produced at less than $2.5/gal or $3.0/gge (gal gasoline equivalent), which can compete favorably with biobutanol ($4.5/gal) produced from corn or sugars in ABE fermentation and bioethanol ($2.0/gal but 25% less energy) currently dominating the biofuel market.

TABLE 2
Stoichiometric equations and mass balance for the microbial consortia grown
on glucose.

| Item | Description |
|---|---|
| Chemistry | a. LAB fermentation: Lactic acid production from glucose |
| | Glucose (C₆H₁₂O₆) + 2 ADP + 2 Pi → 2 Lactic acid (C₃H₆O3) + 2 ATP |
| | b. Acetogenic fermentation: Acetic acid production from lactic acid and |
| | formic acid |
| | Lactic acid (C₃H₆O₃) + 0.5 ADP + 0.5 Pi → 1.5 Acetic acid (C₂H₄O₂) + 0.5 |
| | ATP |
| | Formic acid (CH₂O₂) + 2 H⁺ + 0.5 ATP → 0.5 Acetic acid (C₂H₄O₂) + H₂O + |
| | 0.5 ADP + 0.5 Pi |
| | c. Mixotrophic acetogenic fermentation: Acetate from lactate and formate |
| | Lactic acid (C₃H₆O₃) + Formic acid (CH₂O₂) + 2 H⁺ → 2 Acetic acid |
| | (C₂H₄O₂) + H₂O |
| | d. Butanol production from glucose by C. tyrobutyricum (via AdhE2) |
| | Glucose (C₆H₁₂O₆) + 2 NADH + 2 H⁺ + 2 ADP + 2 Pi → Butanol (C₄H₁₀O) + 2 |
| | CO₂ + 2 H₂ + H₂O + 2 NAD⁺ + 2 ATP |
| | e. Butyric acid production from glucose by C. tyrobutyricum |
| | Glucose (C₆H₁₂O₆) + 3 ADP + 3 Pi → Butyric acid (C₄H8O₂) + 2 CO₂ + 2 H₂ + |
| | 3 ATP |
| | f. Butyric acid production from acetic acid |
| | 2 Acetic acid (C₂H₄O₂) + 2 NADH + 2 H⁺ → Butyric acid (C₄H₈O₂) + 2 H₂O + |
| | 2 NAD⁺ |
| | g. Butanol production from butyric acid (via AOR and BDH) |
| | Butyric acid (C₄H₈O₂) + FdH₂ + NADH + H⁺ → Butanol (C₄H₁₀O) + |
| | H₂O + NAD⁺ + Fd |
| | h. Formic acid from CO₂ and H₂ (via electro-chemical reactions) |
| | CO₂ + H₂ + 2 e⁻ → Formic acid (CH₂O₂) |
| | Reducing equivalents generation from formate (via FDH and electricity) |
| | i. Formic acid (CH₂O₂) + NAD⁺ + 2 H⁺ → NADH + CO₂ + H₂ |
| | j. Fd + 2 H⁺ + 2 e⁻ → FdH₂ |
| | k. Hydrogen from electrolysis |
| | 2 H₂O → 2 H₂ + O₂ |
| Mass | a + d + 2(c + f + g + h): Butanol production from glucose without CO₂ emission |
| Balance | 2 Glucose (C₆H₁₂O₆) + 2 H₂ + 8 NADH + 8 H⁺ + 2 FdH₂ + 4 ADP + 4 Pi → 3 |
| | Butanol (C₄H₁₀O) + 9 H₂O + 8 NAD⁺ + 2 Fd + 4 ATP |
| | Overall, one mol glucose can be converted to 1.5 mol butanol with 4 NADH, 1 |
| | FdH₂ and 1 H₂. Thus, a total of 12 reducing or electron equivalents (2 e- for each |
| | formate, NADH, and FdH₂ generated in reactions h, i, and j, respectively) must |
| | be supplied to the system with renewable electricity. The additional hydrogen |
| | can be provided from electrolysis (reaction k). The max. mass yield of butanol |
| | from glucose is 0.618 g/g or 1.619 kg of glucose will be used for each kg butanol |
| | produced. Assuming ~10% of glucose was used in cell growth, the net butanol |
| | yield would be ~0.56 g/g or ~1.8 kg of glucose for each kg butanol produced. |
| Specific Energy Ratio | ${U}_{Butanol} / {U}_{glucose} = \frac{36 MJ / kg * 1 kg}{15.5 MJ / kg * 1.619 kg} = 1.435 > 1$ |

### Electrochemical Reduction of CO₂ to Formate

CO₂ released in sugar fermentation can be captured by converting it electrochemically to formate or formic acid. In electrochemical reduction, electrons are transferred to CO₂ at the cathode (CO₂+H₂O+2 e⁻→HCOO⁻+OH⁻) and electrolysis of water releases electrons to the anode (H₂O→2 H⁺+0.5 O₂+2 e⁻). The overall reaction (CO₂+H₂O→HCOOH+0.5 O₂) also releases 0.5 O₂. Only electricity and water are needed to produce formate from CO₂, which is estimated at a cost of ˜$200/ton or lower. Formate can also be produced by direct catalytical CO₂ hydrogenation, which however requires relatively expensive catalysts. A life cycle assessment (LCA) shows the electrochemical reduction approach has smaller carbon footprints compared to direct CO₂ hydrogenation. LCA also shows the electrochemical reduction of CO₂ has lower environmental impacts for scale-up compared to the conventional formate production from methyl formate. A techno-economic analysis also shows electrochemical reduction is feasible for large scale production (100 tons/day CO₂ consumption). However, there is a risk in using formate in fermentation as its toxicity to some microbes is relatively high. The toxicity risk can be mitigated by integrating electrochemical formate production from CO₂ with the fermentation for simultaneous formate consumption as demonstrated with Ralstonia eutropha for butanol production. Shielding the anode with a porous ceramic cup also avoided reactive oxygen and nitrogen species from inhibiting microbial metabolism. One can control the formate concentration in the medium at a non-toxic level by feeding formate to the fermenter periodically. Alternatively, cells can be adapted to tolerate formate to a higher concentration via evolutionary engineering, which has been used to adapt clostridia to tolerate 2 to 3-fold higher concentrations of butyrate and butanol. In summary, an electrochemical reduction process can be used to convert CO₂ to formate under conditions compatible to fermentation. The produced formate can then be used in fermentation as energy/electron carrier. Cathodic materials, such as Pb-based and carbon nanotubes, suitable for large-scale industrial operation working at different conditions (pH, current density, temperature) can achieve a formate faradaic efficiency of >80%.

### Lactic Acid Bacteria

Lactic acid bacteria (LAB) commonly used in industrial production of lactic acid are Gram-positive, anaerobic, heterotrophic, and extremely fastidious. LAB consist of several genera in Firmicutes phylum including Lactococcus, Lactobacillus, Streptococcus, Leuconostoc, and Pediococcus. Their growth utilizes organic carbon, such as glucose, sucrose and lactose, and complex nutrients including amino acids, peptides, nucleic acid derivatives, vitamins, mineral salts, fatty acids, and fatty acid esters. As anaerobes, LAB do not use oxygen as the electron receptor, but they possess peroxidase and can grow in the presence of oxygen. Lactic acid is the major product with acetic acid, ethanol, and acetoin also produced as by-products by some LAB. Homofermentative LAB or homolactic acid bacteria, including Lactococcus lactis, Streptococcus lactis, Lactococcus plantarum, Lactobacillus delbrueckii, and Lactobacillus bulgaricus, convert one mole of glucose to two moles of lactic acid through Embden-Meyerhof-Parnar glycolytic pathway, with a product yield of ˜0.95 g/g glucose metabolized in the fermentation. Glucose, lactose, and sucrose are the common carbon sources used in industrial lactic acid fermentation. Some LAB can use starch directly, but none can ferment pentoses or gluconate. Most LAB strains grow at temperatures between 25 to 45° C. and pH between 4.5 and 7.5, with the optimal temperature of 32-37° C. and optimal pH of 5.5-6.5.

### Acetogens and Acetic Acid Fermentation

Acetogens are obligately anaerobic bacteria which can grow both heterotrophically on organic substrates and autotrophically on CO₂ and H₂ using the Wood-Ljungdahl pathway. Acetogens are very diverse and widely distributed among several members of bacterial phyla, including Spirochaetes, Firmicutes (e.g., Clostridia), Chloroflexi, and Proteobacteria. There are over 100 acetogenic species in 22 genera isolated from a variety of habitats (e.g., soils, sediments, sludge, and the intestinal tracts of many animals, including humans and termites). Homoacetogens, which produce only acetate as the main fermentation product without releasing any CO₂, attract high interest for their potential use in acetic acid production because of their high acetate yield and ability to utilize CO₂ and H₂ to form acetate. Nearly 100% of the substrate carbon can be recovered in acetic acid by homoacetogenic fermentation. Clostridium and Acetobacterium are the most studied homoacetogenic genera. Thermophilic homoacetogens such as M. thermoacetica, M. thermoautotrophica and Acetogenium kivui have the optimal growth temperature between 60° C. and 70° C., whereas mesophilic homoacetogens such as C. aceticum, C. formicoaceticum, C. magnum, A. woodii, and A. carbinolicum have the optimal growth temperature between 30° C. and 37° C., which are listed in the following Table 3 with their respective growth substrates and optimal temperature and pH.

TABLE 3
Homoacetogens and their optimal growth conditions and substrates.

| | | Optimal |
|---|---|---|
| | | Temp. |
| Microorganisms | Carbon Substrates | & pH |
| Moorella | Fructose, pyruvate, glucose, | 60° C., 7.0 |
| thermoacetica | xylose, lactate, CO₂/ | |
| | H₂/CO | |
| M. thermoautotrophica | Fructose, glucose, galactose, | 60° C., 5.7 |
| (C. | glycerol, methanol, lactate, | |
| thermoautotrophicum) | formate, CO₂/H₂/CO | |
| Clostridium aceticum | Fructose, pyruvate, CO₂/H₂/CO | 30° C., 8.3 |
| C. formicoaceticum | Fructose, pyruvate, lactate, | 37° C., 7.6 |
| | formate, glycerol, CO₂/ | |
| | H₂/CO | |
| C. magnum | Fructose, glucose, sucrose, | 30° C., 7.0 |
| | xylose, citrate, malate | |
| Acetobacterium | Fructose, glucose, lactate, | 30° C., 6.7 |
| woodii | formate, glycerol, CO₂/ | |
| | H₂/CO | |
| Acetobacterium | Fructose, glucose, pyruvate, | 27° C., 7.0 |
| carbinolicum | lactate, formate, aliphatic alcohols | |
| | C₁-C₅, CO₂/H₂ | |
| Acetogenium kivui | Fructose, glucose, mannose, | 66° C., 6.4 |
| | pyruvate, formate, CO/H₂ | |

Most of homoacetogens can utilize a wide variety of carbon sources, including one-carbon compounds such as CO₂, CO, formate and methanol, sugars such as fructose and glucose, and other compounds such as lactate, glycerol and pyruvate for cell growth and acetate production. M. thermoacetica (C. thermoaceticum) can convert glucose, xylose, and cellulose to acetic acid. A mutant strain of C. thermoaceticum was able to produce acetate at a high concentration of 10% (w/v) in fed-batch fermentation with cell recycle, which gave a reactor productivity of ˜0.8 g/L·h and acetate yield of ˜0.8 g/g glucose using a rich synthetic medium with yeast extract. C. formicoaceticum cannot use glucose but was able to produce acetic acid from fructose, lactate, formate, and glycerol. With cells immobilized in a fibrous bed bioreactor (FBB), a high acetic acid yield of ˜1.0 g/g fructose, a final acetate concentration of ˜78 g/L and productivity of ˜0.95 g/L·h were achieved in fed-batch fermentation at pH 7.6 and 37° C. Acetobacterium BR-446 was able to produce acetic acid from CO₂ and H₂ at a high productivity of 6.2 g/L·h in a continuous fermentation with membrane filtration for high cell density. Among the known acetogens, A. woodii has the highest ability to use CO₂/H₂ for acetate production. A production of ˜35 g/L acetate from CO₂/H₂ at a volumetric productivity of ˜0.8 g/L·h was reported for an engineered strain of A. woodii cultured in a stirred tank reactor. The stoichiometric equations for acetate (CH₃COOH) production from sugar (C₆H₁₂O₆), lactate (CH₃CHOHCOOH), and formate (HCOOH) by homoacetogen are given below with their respective theoretical yields:
Glucose or Fructose: C₆H₁₂O₆→3 CH₃COOH (Theoretical yield: 1.0 g/g)
Lactic Acid: CH₃CHOHCOOH→1.5 CH₃COOH (Theoretical yield: 1.0 g/g)
Formic Acid: HCOOH+H₂→0.5 CH₃COOH+H₂O (Theoretical yield: 0.65 g/g)

In addition to acetate, some acetogens including C. autoethanogenum, C. carboxidivorans, C. ragsdalei, and C. ljungdahlii can also produce ethanol, butyrate, and butanol from CO₂ and H₂. These carboxydotrophs can also grow heterotrophically on glucose and other organic matters such as glycerol. Furthermore, formate is the preferred single-carbon substrate than CO₂ for most of carboxydotrophs.

Mixotrophic Fermentation with Acetogen for Recapturing CO₂ and H₂

About one third of the substrate carbon in glucose fermentation is lost as CO₂, which not only limits product yield but also causes environmental concern due to the release of CO₂, a major greenhouse gas (GHG). It is thus desirable to capture and utilize the fermentation-produced gases (CO₂ and H₂) for butanol production. Many acetogens, including Acetobacterium woodii, C. aceticum, C. autoethanogenum, C. ljungdahlii, and Eubacterium limosum, can convert or fix CO₂ with H₂ or CO as the energy source and electron donor to acetyl-CoA via the Wood-Ljungdahl (W-J) pathway, which is known as the most energy-efficient pathway for CO₂ fixation. Although mixotrophic acetogens can concurrently utilize sugars and gases (CO₂ and H₂) for acids (mainly acetate) and alcohols (mainly ethanol) production, their industrial application is limited by low product yield and productivity largely due to poor gas solubility and extremely high sensitivity to oxygen, which is highly toxic to acetogens.

Similar to more well studied mixotrophic homoacetogens, C. formicoaceticum naturally converts various carbon sources, including fructose, glycerol, lactate and formate, to acetate as the sole fermentation product with a >90% (w/w) carbon recovery yield without losing much carbon to cell biomass or CO₂. To achieve zero or negative CO₂ emission, formate (produced from CO₂) must be fully uptaken by C. formicoaceticum. However, C. formicoaceticum grew poorly on CO₂ or formate as the sole carbon source, because 0.5 mol ATP was used for each mol formate consumed for acetate production, leading to energy imbalance in cells. On the contrary, each mol lactate converted to acetate can generate 0.5 mol ATP. Thus, energy balance is maintained in mixotrophic fermentation with equal molar lactate and formate as co-substrates, producing 2 mol acetate without losing any substrate carbon to CO₂. Therefore, using formate with lactate as a co-substrate in the mixotrophic fermentation by C. formicoaceticum or an acetogen would be an efficient way to produce acetate, which can be further converted to butyrate and then butanol by solventogenic Clostridia such as C. tyrobutyricum, thus providing an effective process for recapturing CO₂ and H₂ for butanol production.

The stoichiometric equations for acetate production from lactate and formate (HCOOH) in a mixotrophic fermentation by a homoacetogen such as C. formicoaceticum are listed below:
Mixotrophic fermentation: Lactate→1.5 Acetate
   Formate→0.5 Acetate
Overall: Lactate+Formate→2 Acetate

### Example 1: Acetate Production from Various Carbon Sources by C. Formicoaceticum

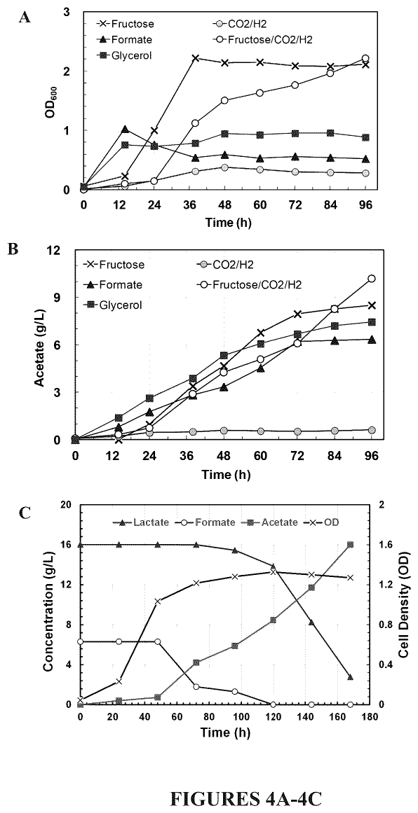

C. formicoaceticum can produce acetate from cheap and abundant industrial byproducts, such as glycerol and syngas. The fermentation kinetics of C. formicoaceticum using fructose, glycerol, and formate as a substrate, respectively, were studied under anaerobic conditions at a growth pH of 7.6 in serum bottles (FIGS. 4A-4B). Cells grew on fructose robustly and rapidly reached the stationary phase in 36 h, and acetate was the sole fermentation product with a high yield of 0.91 g/g without losing any carbon to CO₂. C. formicoaceticum can also utilize glycerol or formate as the carbon source for growth, although at a slower growth rate compared to that of utilizing fructose, with acetate as the sole product, at a yield of ˜0.8 g/g and ˜0.6 g/g, respectively. C. formicoaceticum grew poorly on CO₂ and H₂. Further experiments in serum bottles were carried out for the mixotrophic fermentation with formate and lactate as co-substrates for C. formicoaceticum and the results showed that acetate yield of 0.9 g/g representing a 95.5% recovery of the substrate carbon in acetate was obtained from 5.4 g/L lactate and 2.1 g/L formate. This meant only a small amount of substrate carbon was used in growing cell biomass and no CO₂ was released in the fermentation. The mixotrophic fermentation with formate and lactate as co-substrates at higher initial concentrations was also studied in bioreactors with pH controlled at ˜7.5 (FIG. 4C). With initially ˜10 g/L lactate and ˜4 g/L formate, acetate titer increased to ˜12 g/L while the yield was ˜1.06 g/g. When lactate was further increased to ˜16 g/L and formate to ˜6 g/L, the acetate titer also increased to ˜16 g/L with ˜0.82 g/g yield. The results from various mixotrophic fermentations in serum bottles and bioreactors are summarized in the following Table 4. In general, an overall acetate yield of >0.9 g/g lactate and >90% carbon recovery from both lactate and formate were achieved in the mixotrophic fermentation.

TABLE 4
Mixotrophic fermentation of lactate and formate by C. formicoaceticum.

| | | | | Acetate | Carbon | |
|---|---|---|---|---|---|---|
| | Lactate | Formate | Acetate | Yield | recovery | Productivity |
| Batch | (g/L) | (g/L) | (g/L) | (g/g) | % | (g/L · h) |
| Serum bottle 1 | 5.4 | 2.1 | 6.7 | 0.90 | 95.5 | 0.14 |
| Serum bottle 2 | 5.6 | 2.8 | 6.2 | 0.75 | 86.6 | 0.12 |
| Serum bottle 3 | 5.9 | 5.1 | 7.5 | 0.68 | 82.5 | 0.16 |
| Serum bottle 4 | 11.0 | 6.6 | 6.7 | 0.59 | 65.7 | 0.08 |
| Serum bottle 5 | 14.3 | 4.1 | 8.4 | 0.69 | 73.6 | 0.18 |
| Bioreactor 1 | 10.9 | 4.1 | 12.3 | 1.06⁺ | 121.0⁺ | 0.24 |
| Bioreactor 2 | 16.0 | 6.3 | 16.0 | 0.82 | 92.0 | 0.16 |

*Acetate yield: Overall yield from total lactate and formate consumed; Carbon recovery %: percentage of substrate carbon recovered in the product acetic acid; Productivity: acetate production during the exponential phase.
⁺The higher than 100% carbon recovery and >1.0 acetate yield could be attributed to the errors in HPLC analysis of fermentation samples.

### Acetate Production in Cocultured Fermentation of LAB and Acetogen

Microbes are mutually interactive and, when assembled as symbiotic ecosystems, can give rise to an improved overall performance for fermentation than mono-species. LAB, such as L. lactis, can better tolerate oxygen and their growth would remove trace oxygen dissolved in the culture medium; as a result, LAB will alter the medium to the state better suited for growing the strictly anaerobic acetogen. Meanwhile, toxic metabolites such as lactic acid and ammonia produced by LAB can be used as carbon and nitrogen sources, respectively, by the acetogen to produce acetate. Thus, a properly composed consortium of strains would allow them to grow to a well-balanced population. Previous studies have shown that C. formicoaceticum cocultured with LAB produced acetate from sugars, including lactose and glucose, with lactate as the intermediary product, at a >0.95 g/g yield. Thus, the coculture of LAB and acetogen will produce acetate from glucose and formate with a ˜100% carbon recovery rate. The cocultured fermentation can be optimized by controlling the growth and lactate production rates by LAB to balance with the growth and lactate consumption rates by the acetogen. As illustrated below, 3 moles of acetate can be produced from each mole of glucose fermented by the homoacetogen co-cultured with homolactic acid bacteria. Likewise, 3.5 moles of acetate can be produced from one mole of glucose and one mole of formate in the cocultured fermentation.
Glucose→2 Lactate→3 Acetate
Glucose+Formate→2 Lactate+Formate→3.5 Acetate

### Example 2: Acetate Production from Sugar by L. lactis and C. Formicoaceticum in a Coculture

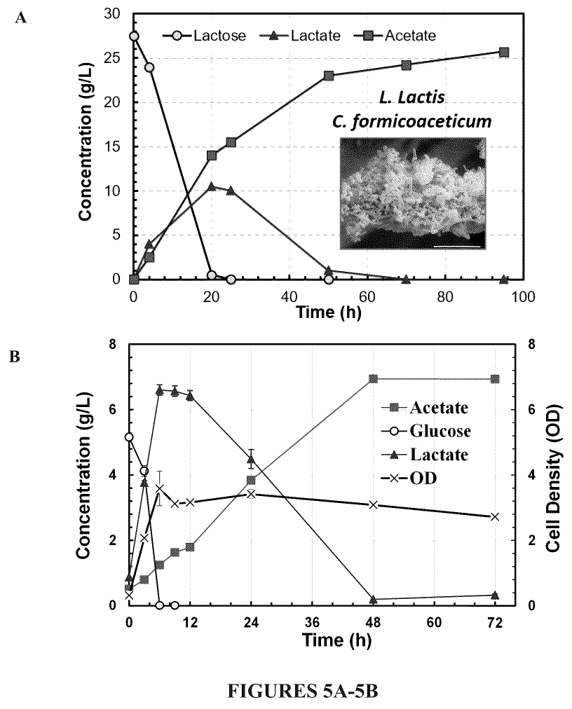

Acetate production from lactose in fermentation with L. lactis and C. formicoaceticum co-immobilized in a fibrous bed bioreactor was first studied. About 25.5 g/L acetate was produced from 27.5 g/L lactose in 95 h (FIG. 5A). Lactate produced as an intermediary metabolite by L. lactis was completely converted to acetate by C. formicoaceticum by 95 h with a product yield of ˜0.93 g/g lactose. Similar results were obtained in glucose fermentation with C. formicoaceticum co-cultured with L. plantarum or L. lactis (FIG. 5B). Glucose was rapidly converted to lactate by LAB within 9 h. Meanwhile, acetate was coproduced by C. formicoaceticum from lactate and acetate production continued until all lactate was consumed in 48 h. Overall, ˜7 g/L acetate was produced from glucose and lactate at a productivity of ˜0.15 g/L·h and acetate yield of ˜0.87 g/g for C. formicoaceticum co-cultured with L. lactis and ˜0.96 g/g for the co-culture with L. plantarum, respectively. The productivity and yield from the co-cultured fermentation were comparable to those obtained in the mixotrophic fermentation by C. formicoaceticum grown on lactate and formate. These experiments confirmed that a high acetate yield of >0.9 g/g substrate can be obtained in the co-cultured fermentation.

Butanol Production by Engineered C. tyrobutyricum

Historically, n-butanol has been produced from starch and sugar-based feedstocks in industrial acetone-butanol-ethanol (ABE) fermentation by solventogenic clostridia (such as Clostridium acetobutylicum, Clostridium beijerinkii, Clostridium saccharolyticum, and Clostridium saccharoperbutylacetonicum) which is limited by low butanol titer (˜12 g/L), yield (˜0.2 g/g), and productivity (˜0.25 g/L·h). Furthermore, the ABE fermentation is unstable and difficult to control due to clostridia's complex life cycle involving acidogenesis, solventogenesis, and sporulation that are highly regulated by multiple gene regulators involving various kinases, transcription factors, and interlocking signal transduction pathways. Under stress, clostridia sporulate and halt their metabolism, limiting their ability to produce butanol at desirable titers, rates, and yields and longevity for continuous operation. To alleviate these problems, a number of solventogenic clostridial strains have been developed by metabolic and evolutionary engineering for n-butanol production. For example, solventogenic Clostridium strains were developed by introducing n-butanol biosynthesis pathway in non-solventogenic acid-forming Clostridium tyrobutyricum and Clostridium cellulovorans, which have relatively simple metabolic pathways with high flux toward butyryl-CoA, the precursor for n-butanol biosynthesis, and high butanol tolerance.

Most butyric acid-producing clostridia ferment glucose, xylose, lactose, starch, and glycerol for cell growth and butyric acid production. C. butyricum can also produce 1,3-propanediol from glycerol. Other substrates such as cellulose and CO₂ can also be used for butyrate production. C. carboxidivorans and Butyribacterium methylotrophicum can utilize CO, CO₂ and H₂ to produce butyric acid. C. cellulovorans, C. polysaccharolyticum and C. populeti can convert cellulose to butyrate as the major fermentation product, whereas C. kluyveri can produce butyric acid as a major product from ethanol and acetate as substrates. These Clostridia can be used in the consortia to produce butyric acid and butanol. Most butyric acid bacteria have an effective growth temperature between 3° and 37° C. and pH between 5.5 and 6.5. The thermophilic C. thermobutyricum grows effectively at ˜60° C.

C. tyrobutyricum is frequently used for butyric acid production because of its high metabolic flux toward butyryl-CoA and high butyric acid tolerance. C. tyrobutyricum uses CoA transferase (CoAT) encoded by cat1, instead of phosphotransbutyrylase (ptb) and butyrate kinase (buk), to convert butyryl-CoA to butyrate, which transfers the CoA to acetate to form acetyl-CoA, thus allowing the reassimilation of acetate. Since acetate, a major byproduct in butyrate fermentation, can be re-assimilated to generate butyrate via the CoAT pathway, acetate production can be reduced to minimum with butyrate as the main or only fermentation product. C. tyrobutyricum has also been engineered as a superior host for n-butanol production, leveraging its high metabolic flux toward butyryl-CoA and tolerance to butanol (>15 g/L). C. tyrobutyricum Δack-adhE2 overexpressing adhE2, encoding a bifunctional aldehyde/alcohol dehydrogenase (ALD/ADH) from Clostridium acetobutylicum produced n-butanol as the main product from glucose (via butyryl-CoA) with butyrate as a byproduct. Unlike the conventional solventogenic clostridia in ABE fermentation, the heterologous n-butanol biosynthesis pathway in the engineered C. tyrobutyricum is easier to control as the fermentation does not involve a phase transition from acidogenesis to solventogenesis. However, the co-production of CO₂ and butyrate limits its butanol yield to ˜0.3 g/g glucose.

An indigenous Type I-B CRISPR-Cas system was developed and used to insert adhE2 and replace cat1 on the genome in C. tyrobutyricum. The mutant C. tyrobutyricum Δcat1: adhE2 produced little butyrate and more n-butanol as the main product, but acetate and ethanol were also produced in higher amounts. Consequently, butanol yield was still limited to ˜0.30 g/g glucose. The increased acetate production can be attributed to the knockout of cat1, which debilitates the reassimilation of acetate. The increased ethanol production can be attributed to the lack of additional 2 mol NADH required for butanol biosynthesis from butyryl-CoA, which would cause redox imbalance. Therefore, to increase butanol production in C. tyrobutyricum, one must increase intracellular NADH availability and further convert butyrate to butanol.

To increase butanol biosynthesis via the ALD-ADH pathway, external reducing equivalents can be provided by supplying formate to solventogenic clostridia overexpressing a heterologous formate dehydrogenase (FDH) gene, such as fdh from C. beijerinckii or other species. With FDH, formate can provide two electrons to NAD(P)H required for the reactions from butyryl-CoA to butanol, thus increasing butanol (over butyric acid) production in the fermentation.

The conversion of pyruvate to acetyl-CoA is coupled with the reduction of ferredoxin to reduced ferredoxin Fed-H₂, which is re-oxidized by either releasing H₂ via iron hydrogenase or transferring electrons to NAD(P)³⁰ , thus regenerating NAD(P)H, via ferredoxin-NAD(P)⁺ oxidoreductase (FNR). Thus, increasing FNR activity can increase NAD(P)H availability and provide additional reducing equivalents for butanol biosynthesis. Therefore, overexpressing a heterologous FNR gene, such as fnr from C. acetobutylicum, in engineered Clostridia with adhE2 can increase butanol biosynthesis. In addition, knocking out the redox-sensing transcriptional repressor gene (rexA) and overexpressing a NADP+-dependent 3-hydroxybutyryl-CoA dehydrogenase (hbd) in C. Acetobutylicum has been shown to increase butanol biosynthesis from glucose.

### Example 3: Effects of Overexpressing Hbd on Butanol Production by C. tyrobutyricum

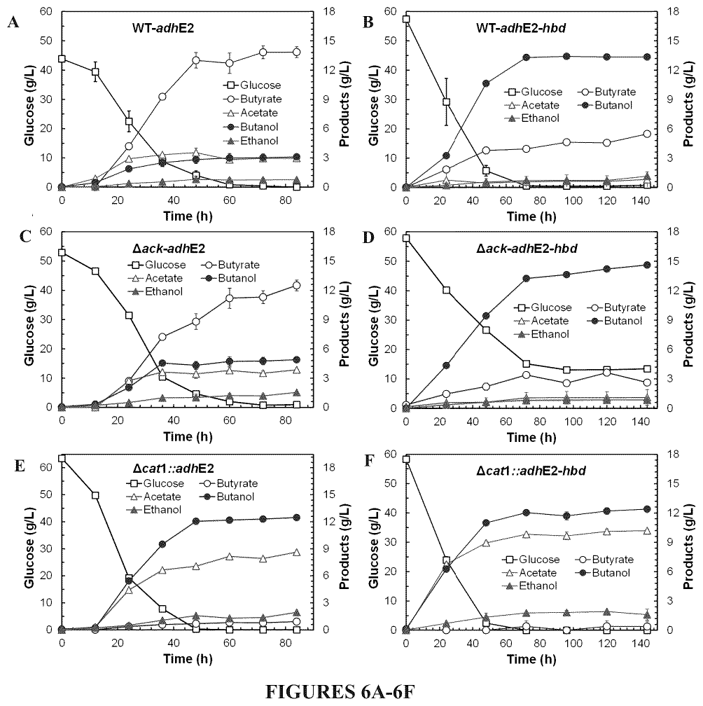

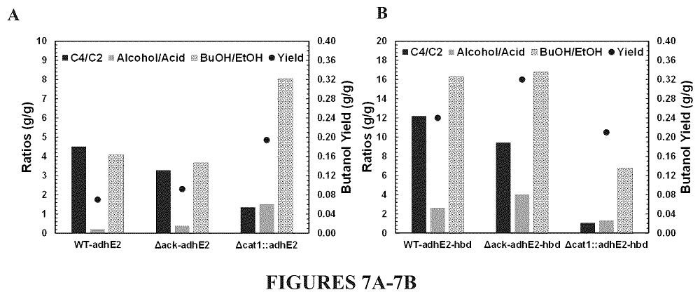

C. tyrobutyricum strains WT-adhE2-hbd, Δcat1::adhE2-hbd, and Δack-adhE2-hbd were constructed to overexpress hbd (from Clostridium kluyveri) encoding the NADPH-dependent β-hydroxybutyryl-CoA dehydrogenase, which used NADPH instead of NADH as the cofactor in the reaction from acetoacetyl-CoA to 3-hydroxybutyryl-CoA. These mutant strains and their parental strains (without hbd) were grown in Clostridia Growth Medium (CGM) containing glucose as the carbon source in serum bottles at 37° C. The medium pH was buffered at ˜5.5 with CaCO₃. The time course data on glucose, butanol, ethanol, acetate and butyrate in batch fermentations are shown in FIGS. 6A-6F. The mutant strains with cat1 knockout had significantly reduced butyrate but much higher acetate production. Δcat1::adhE2 produced little butyrate but high acetate, whereas Δack-adhE2 produced less acetate but more butyrate. Compared to the parental strains overexpressing only adhE2, mutants also overexpressing hbd gave much higher butanol production with a butanol yield as high as 0.32 g/g glucose consumed, which was more than 50% increase. Also, much higher butanol/ethanol, alcohol/acid, and C4/C2 ratios were obtained in the fermentations with the hbd-overexpressing mutants (FIGS. 7A-7B), confirming that overexpressing hbd significantly increased intracellular NADH pool for butanol biosynthesis.

### Example 4: Effects of pH on Butanol Production by C. tyrobutyricum

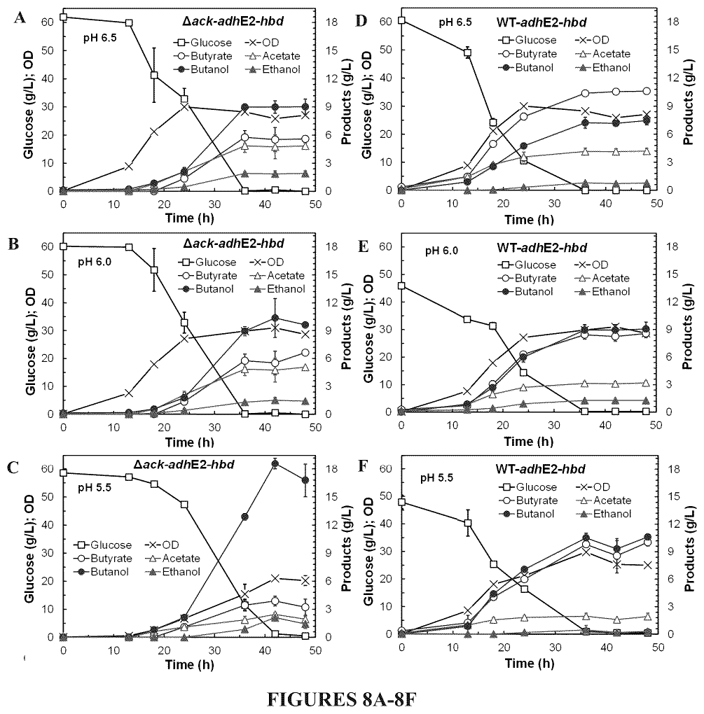

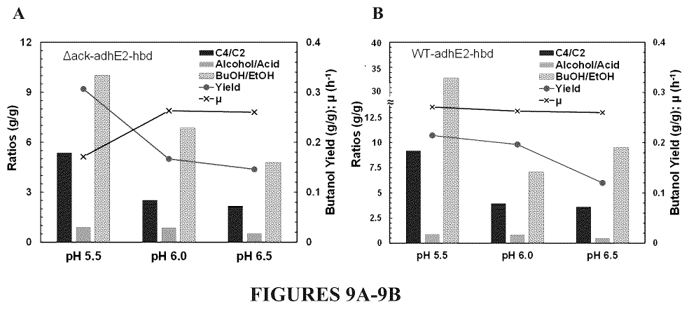

C. tyrobutyricum strains WT-adhE2-hbd and Δack-adhE2-hbd were cultured in Clostridia Growth Medium (CGM) containing glucose as the carbon source in bioreactors with pH controlled at 5.5, 6.0, and 6.5, respectively, with Ca(OH)₂ and temperature at 37° C. The time course data on cell density (OD), glucose, butanol, ethanol, acetate and butyrate in batch fermentations are shown in FIGS. 8A-8F. In general, higher butanol yield and C4/C2, alcohol/acid, and butanol/ethanol ratios were obtained at the lower pH 5.5 (FIGS. 9A-9B).

### Example 5: Effects of MV on Butanol Production by C. tyrobutyricum

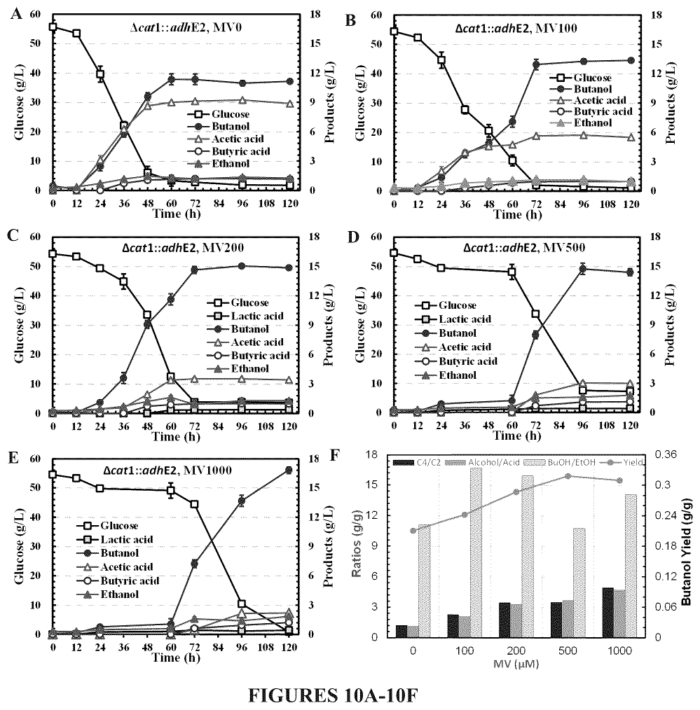

Artificial electron carriers such as methyl viologen, benzyl viologen and neutral red have been used in solventogenic clostridia to increase butanol production through diverting electron flow towards NADH formation accompanied by decreased hydrogen production. An artificial electron carrier, such as methyl viologen (MV) and benzyl viologen (BV), was used to inhibit hydrogen production and increase NADH pool and butanol production by C. tyrobutyricum expressing adhE2. C. tyrobutyricum Δcat1::adhE2 was cultured in Clostridia Growth Medium (CGM) containing glucose as the carbon source in serum bottles at 37° C. The medium pH was buffered at ˜5.5 with CaCO₃. At 24 h after initial seeding the bottles, methyl viologen was added at various concentrations (up to 1000 μM) to study its effects on cell growth and butanol production. The time course data on glucose, butanol, ethanol, acetate, and butyrate in batch fermentations are shown in FIGS. 10A-10F. In general, MV dramatically reduced acetate production and increased butanol production from glucose in these fermentations. Interestingly, with MV at 200 μM and higher, lactate was also produced as a byproduct, indicating that some intracellular NADH generated in glycolysis was consumed for lactate biosynthesis to balance the redox. The highest butanol yield and productivity were obtained at 500 μM MV. Similar results can also be achieved with other electron carriers such as benzyl viologen and neutral red.

### Example 6: Butanol Production from Glucose and Xylose by C. tyrobutyricum in Bioreactor

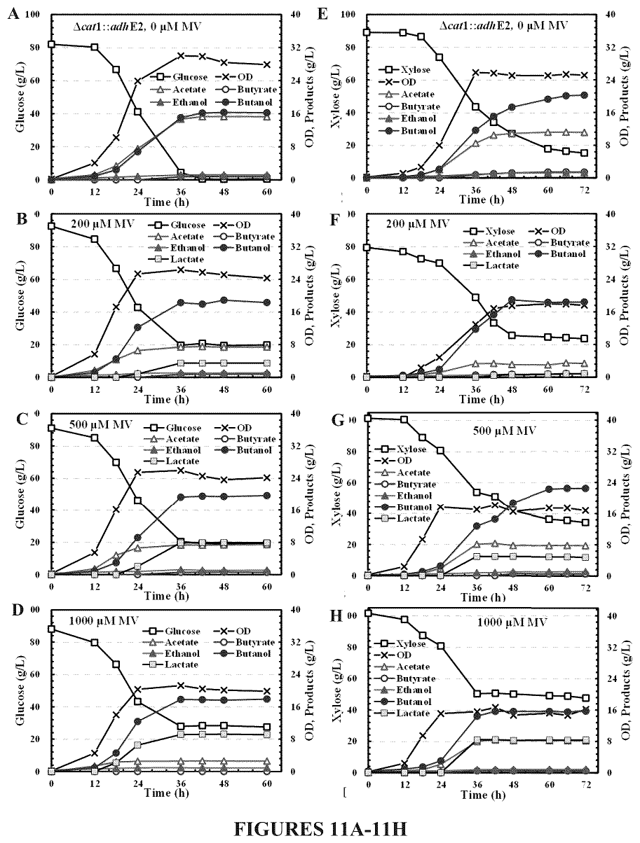

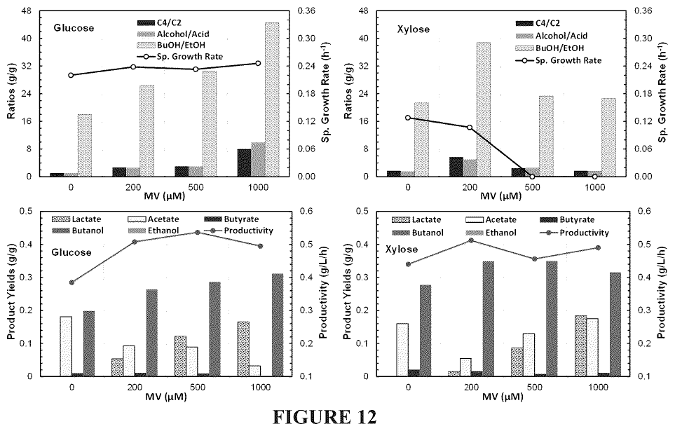

Batch fermentations of glucose and xylose by C. tyrobutyricum Δcat1::adhE2 were studied in bioreactors at pH 6.0 in the presence of methyl viologen (MV) at various concentrations (0 μM, 200 μM, 500 μM, 1000 μM). For glucose fermentation, MV was added at 12 h after inoculation. For xylose fermentation, MV addition was delayed to 24 h for the higher concentrations of 500 μM and 1000 μM, which completely inhibited cell growth and fermentation. The fermentation time course data are shown in FIGS. 11A-11H and the effects of MV on cell growth and fermentation kinetics are illustrated in FIG. 12. With glucose as the substrate, butanol and lactate production increased while acetate and butyrate production decreased with increasing the MV concentration. Consequently, butanol yield, productivity, and C4/C2, alcohol/acid, and butanol/ethanol ratios also increased with MV. Similar trends were also observed with xylose as the substrate at 200 μM MV. However, the higher MV concentrations of 500 μM and 1000 μM completely inhibited cell growth and stopped fermentation upon their addition to the medium at 24 h (FIG. 11G, H). Compared to glucose, xylose as a more reduced substrate produced more butanol, but MV was more toxic to cells grown on xylose. The results also indicate that biomass hydrolysate sugars including glucose and xylose can be used simultaneously and converted to butanol by engineered C. tyrobutyricum expressing adhE2.

### Example 7: Butanol Production from Glucose and Lactate by C. tyrobutyricum

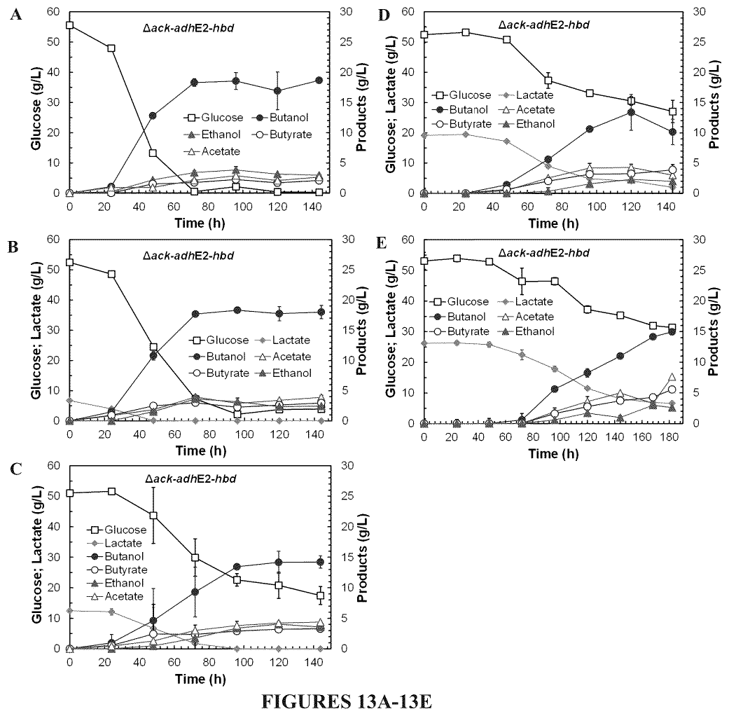

Batch fermentations by C. tyrobutyricum Δack-adhE2-hbd were studied in serum bottles with glucose and lactate as co-substrates at various lactate concentrations from 0 to ˜26 g/L. The fermentation data are shown in FIGS. 13A-13E. In general, glucose and lactate were consumed simultaneously with butanol as the main product at a yield of ˜0.3 g/g substrate consumed. These experiments confirm that lactate can also be used by C. tyrobutyricum for butanol production, suggesting that any lactate produced by a lactic acid bacterium cocultured with C. tyrobutyricum can also be converted to butanol.

### Example 8: Batch Fermentation with Acetate as a Co-Substrate

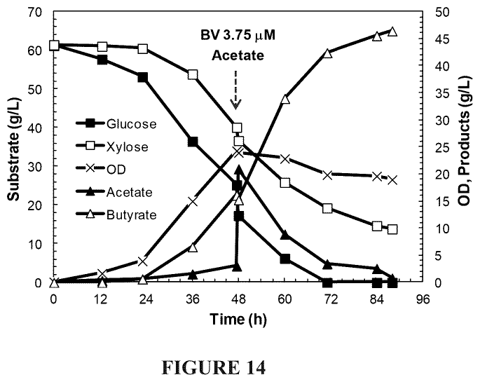

Batch fermentation in a stirred-tank bioreactor with C. tyrobutyricum grown on glucose and xylose at pH 6.0 was studied with the addition of ˜18 g/L acetate and 3.75 μM benzyl viologen (BV) at ˜48 h. The fermentation time-course data are shown in FIG. 14. With BV and acetate addition, C. tyrobutyricum converted glucose, xylose, and acetate simultaneously to butyrate as the sole fermentation product with close to 100% purity at an overall yield of ˜0.48 g/g substrate. Almost all glucose and acetate (both produced before BV addition and externally added) were consumed to produce butyrate, reaching a high concentration of ˜46 g/L. Butyric acid yield from sugars increased from 0.30 g/g before BV addition to 0.52 g/g from both sugars and acetate consumed in the presence of 3.75 μM BV. The theoretical yield of butyric acid from glucose in the fermentation is 0.8 mol/mol (0.39 g/g) when acetate (0.4 mol/mol) and carbon dioxide (2 mol/mol) are also produced as co-products. In contrast, butyrate formation from acetate has a theoretical yield of 0.73 g/g (0.5 mol/mol), which is 87.2% higher than that from glucose, because no substrate carbon is lost in carbon dioxide. In addition to the higher butyrate yield, butyrate was produced as the only main product in the fermentation with a high acid purity of 98.3%, which was much higher than ˜80% in typical butyric acid fermentation. As observed in the experiment, acetate, both intrinsically produced and externally provided during the fermentation, were completely assimilated to butyrate by C. tyrobutyricum when there was sufficient reducing power or NADH as mediated by the addition of BV.

BV inhibits hydrogenase and thus prevents electrons removed from oxidation of organic substrates (e.g., glucose) from transferring to protons (H⁺) as a final electron acceptor. Consequently, electrons are transferred to NADH that must be oxidized to allow the fermentation or glucose metabolism to continue. Meanwhile, less ATP would be generated for cell growth when acetate biosynthesis via the reactions catalyzed by phosphotransacetylase and acetate kinase were inhibited by exogenous acetate. Furthermore, C. tyrobutyricum uses CoA transferase to convert butyryl-CoA to butyrate, which transfers the CoA to acetate to form acetyl-CoA, thus allowing the reassimilation of acetate. Thus, acetate assimilation should happen simultaneously with glucose consumption, which generates ATP and electrons, and butyrate biosynthesis, which consumes electrons or NADH. Therefore, additional NADH supply via BV addition is beneficial to push the carbon flux from acetyl-CoA toward butyryl-CoA, which in turn accelerates acetate assimilation and butyrate production, resulting in higher productivity, yield, and selectivity of butyric acid in the fermentation.

### Example 9: Butanol Production from Lignocellulose Sugars by C. tyrobutyricum

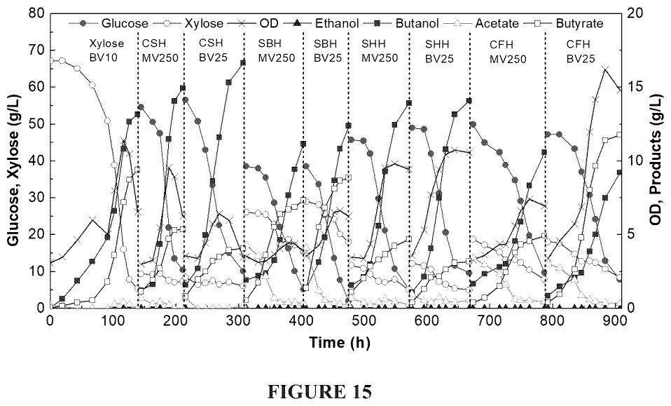

Cells of C. tyrobutyricum Δack-adhE2 were immobilized in a fibrous-bed bioreactor (a glass column packed with a spiral wound fibrous matrix; working volume, 400 mL) connected to a stirred-tank reactor (1.5 L working volume) with pH controlled at 6.0 with saturated NH₄OH solution and at 37° C. The bioreactor was operated in a repeated-batch (RB) mode using various lignocellulose hydrolysates containing glucose, xylose, and acetate as substrates for butanol production. Batch fermentations with hydrolysates of cotton stalk (CSH), sugarcane bagasse (SBH), soybean hull (SHH), and corn fiber (CFH) were operated sequentially to evaluate fermentation performance, with the results shown in FIG. 15. In the study, xylose was used as the substrate with 10 μM BV in the first batch (RB 1), whereas CSH, SBH, SHH, or CFH with 250 μM MV or 25 μM BV were used in subsequent batches (RB 2-9). A high butanol titer of >15 g/L was obtained in CSH fermentations (RB 2-3). SBH (RB 4-5) and SHH (RB 6-7) fermentations also gave good butanol production (12-14 g/L) comparable to RBI (the control) with xylose as substrate, whereas a lower butanol production (˜10 g/L) was obtained in CFH fermentations (RB 8-9). Almost no ethanol or acetate was produced in these fermentations. Furthermore, acetate present in the hydrolysates was also used as carbon source by C. tyrobutyricum Δack-adhE2. This study clearly demonstrated that butanol and butyrate as a byproduct can be produced from glucose, xylose, and acetate present in lignocellulose hydrolysates by engineered C. tyrobutyricum overexpressing adhE2 such as the strain Δack-adhE2. A butanol yield of ˜0.3 g/g substrate was obtained from CSH, 0.22-0.28 g/g from SBH and SHH, and 0.18 g/g from CFH. The significantly lower butanol yield from CFH was attributed to the large amounts of butyric acid also produced in the fermentation. One can thus obtain a higher butanol yield of >0.3 g/g if butyric acid production was limited to a lower level such as in the fermentation using the strain Δack-adhE2-hbd. It is also possible to convert butyric acid to butanol with AOR or carboxylic acid reductase (CAR) also expressed in C. tyrobutyricum Δack-adhE2.

Butanol Production from Butyrate Via AOR and CAR

Some autotrophic acetogens, such as C. ljungdahlii and C. autoethanogenum, produce ethanol from acetate via aldehyde: ferredoxin oxidoreductase (AOR) to reduce acetate to acetaldehyde and then to ethanol via ADH. C. carboxidivorans produces ethanol and butanol via the AOR-ADH pathway under autotrophic growth and the ALD-ADH pathway under heterotrophic conditions. AOR plays an important role in ATP generation under energy-limited conditions in acetogens. AOR plays an important role in ethanol/acetate formation in acetogens during gas fermentation. Furthermore, exogenous acetate can promote the transcriptional expression of AOR, indicating its close association with acetate conversion to ethanol. However, to maintain redox and energy balance in response to different fermentation conditions, AOR catalyzes the reaction from acetate to acetaldehyde in CO fermentation (high-ATP) but catalyzes the reverse reaction in CO₂/H₂ fermentation (low-ATP). AOR's function also depends on pH, reducing acetate/butyrate to aldehydes with reduced ferredoxin at pH 4.0-5.0 while oxidizing aldehydes to acetate/butyrate with oxidized ferredoxin at pH >5.0. Overexpressing aor or adhE2 in C. carboxidivorans increased its alcohol production from glucose, demonstrating the potential of increasing alcohol production by overexpressing these genes in two different alcohol production pathways. An earlier study has also shown that the resting cells of C. formicoaceticum converted carboxylates to corresponding alcohols in the presence of CO or formate and an electron mediator (MV), confirming the presence of ALD, ADH, and AOR activities in C. formicoaceticum and their ability to convert acetate to ethanol and butyrate to butanol. Thus, overexpression of aor in C. tyrobutyricum Δack-adhE2 can facilitate the reduction of butyrate to butyraldehyde leading to butanol production by ADH.

Butyrate can also be reduced to butyraldehyde by an NADPH-dependent carboxylic acid reductase (CAR). Many carboxylate reductases (CARs) and associated car genes have been isolated from fungi including Neurospora crassa (NcCAR) and bacteria such as Nocardia iowensis (NiCAR) which have emerged as biocatalysts for the selective one-step reduction of carboxylic acids to their corresponding aldehydes. CARs activate the carboxylate substrate at the expense of ATP and use NADPH as the cofactor In catalyzing the reduction reaction. Many bacterial CARs have been cloned, expressed in E. coli, and characterized for their specificity and selectivity for aromatic and aliphatic carboxylates including benzoic acid, short-chain to medium-chain fatty acids, and dicarboxylic acids. Among them, Mycobacterium abscessus CAR (MAB4714), M. chelonae CAR (MCH22995), M. immunogenum CAR (MIM0040), and N. brasiliensis CAR (NBR0960) show good butyrate reduction activity and thus can be used to convert butyrate to butyraldehyde in Clostridia.

### Example 10: Overexpressing AOR for Converting Butyrate to Butanol

To facilitate the reduction of butyrate to butyraldehyde, aldehyde: ferredoxin oxidoreductase (AOR) from acetogenic clostridia such as C. formicoaceticum, C. carboxidivorans, and C. autoethanogenum capable of producing alcohols were overexpressed in C. tyrobutyricum adhE2 and evaluated for its effects on butanol production from glucose and butyrate. The AORs from different Clostridium species were evaluated for their effects on butanol production from butyrate under different conditions (glucose, butyrate, and pH) to optimize AOR efficiency for converting butyrate to butyraldehyde and subsequently to butanol by ADH. Thirteen different genes encoding AOR were isolated from various clostridial species (Table 5) and cloned in C. tyrobutyricum. Recombinant plasmids PMTL82151 containing cat1 promoter and adhE2 and aor were constructed and transformed by conjugation into C. tyrobutyricum wild type strain. Positive transformants were obtained and confirmed by colony PCR. These mutant strains were evaluated for their ability to produce butanol from glucose in fermentation in serum bottles and bioreactors with pH control.

TABLE 5
Aldehyde:ferredoxin oxidoreductase (AOR) from different acetogenic clostridia.

| Source | Locus tag | Gene Label | Annotated enzyme name |
|---|---|---|---|
| C. | CA_RS10430 | aor (Ca) | Aldehyde:ferredoxin |
| acetobutylicum | | | oxidoreductase family |
| C. | CLAU_0089 | aor1 (Cau) | Aldehyde:ferredoxin |
| autoethanogenum | | | oxidoreductase |
| | CLAU_0099 | aor2 (Cau) | Aldehyde:ferredoxin |
| | | | oxidoreductase |
| | CLAU_0452 | aor3 (Cau) | Molybdopterin oxidoreductase |
| | | | (FAD-independent) |
| C. | Ccar_RS12720 | aor1 (Cc) | Aldehyde:ferredoxin |
| carboxidivorans | | | oxidoreductase family |
| C. | BJL90_04450 | aor1 (Cf) | Aldehyde:ferredoxin |
| formicoaceticum | | | oxidoreductase |
| | BJL90_04740 | aor2 (Cf) | Aldehyde:ferredoxin |
| | | | oxidoreductase |
| | BJL90_10120 | aor3 (Cf) | Aldehyde:ferredoxin |
| | | | oxidoreductase |
| C. | CLRAG_29620 | aor1 (Cr) | Aldehyde:ferredoxin |
| ragsdalei | | | oxidoreductase |
| | CLRAG_29710 | aor2 (Cr) | Aldehyde:ferredoxin |
| | | | oxidoreductase family |
| | CLRAG_30540 | aor3 (Cr) | Aldehyde oxidoreductase |
| | CLRAG_17120 | aor4 (Cr) | Aldehyde oxidoreductase |
| C. tyrobutyricum | CTK_C17200 | aor (Ct) | Aldehyde oxidoreductase Mop |

In serum bottles, Ct-adhE2-aor2 (Cf) expressing AOR (aor2) from C. formicoaceticum and Ct-adhE2-aor2 (Cau) expressing AOR (aor3) from C. autoethanogenum showed increased butanol and reduced butyrate production from glucose in the presence of 100 μM MV, mutants expressing aor (Cc) and aor (Ct) showed increased butyrate and reduced butanol production from glucose (Table 6). Interestingly, when a more reduced substrate mannitol was used as the carbon source in the fermentation, significantly higher butanol production was observed. These results suggested that the reduction of acids to aldehydes by AOR was limited by the available reducing equivalent.

TABLE 6
Fermentation kinetics of C. tyrobutyricum expressing various AOR genes from different
acetogenic clostridia in serum bottles with glucose as the carbon source.

| Ct Strain | | | | | | | | | |
|---|---|---|---|---|---|---|---|---|---|
| With | Glucose | Lactic | Acetic | | Butyric | | Butanol | | Alcohol/ |
| 100 μM | consumed | acid | acid | Ethanol | acid | Butanol | Yield | C4/C2 | Acids |
| MV | (g/L) | (g/L) | (g/L) | (g/L) | (g/L) | (g/L) | (g/g) | (g/L) | (g/g) |
| adhE2 | 47.04 | 0.00 | 4.53 | 1.07 | 11.81 | 3.97 | 0.08 | 2.82 | 0.31 |
| | 46.98 | 1.43 | 0.48 | 0.93 | 7.96 | 9.96 | 0.21 | 12.68 | 1.10 |
| adhE2- | 54.61 | 0.0 | 4.56 | 1.13 | 12.00 | 5.72 | 0.10 | 3.11 | 0.41 |
| aor1(Cf) | 54.39 | 2.79 | 0.33 | 1.13 | 10.68 | 9.93 | 0.18 | 14.12 | 0.80 |
| adhE2- | 56.51 | 0.0 | 4.23 | 0.90 | 15.31 | 4.40 | 0.08 | 3.84 | 0.27 |
| aor2(Cf) | 53.98 | 5.14 | 0.37 | 0.71 | 9.22 | 11.70 | 0.22 | 19.37 | 0.84 |
| adhE2- | 54.99 | 0.0 | 2.29 | 1.02 | 16.81 | 3.00 | 0.05 | 5.98 | 0.21 |
| aor3(Cf) | 50.77 | 5.07 | 0.00 | 0.69 | 12.06 | 7.81 | 0.15 | 28.80 | 0.50 |
| adhE2- | 55.61 | 0.0 | 4.09 | 0.67 | 20.94 | 0.42 | 0.01 | 4.49 | 0.04 |
| aor(Cc) | 47.06 | 8.06 | 0.00 | 0.64 | 11.41 | 5.77 | 0.12 | 26.84 | 0.33 |
| adhE2- | 57.16 | 0.0 | 4.15 | 0.63 | 19.45 | 1.29 | 0.02 | 4.34 | 0.08 |
| aor(Ct) | 49.77 | 7.36 | 0.0 | 0.66 | 10.42 | 6.79 | 0.14 | 26.08 | 0.42 |
| adhE2- | 50.74 | 0.0 | 4.49 | 0.75 | 12.57 | 3.53 | 0.07 | 3.07 | 0.25 |
| aor(Ca) | 52.13 | 3.03 | 0.32 | 0.0 | 9.29 | 9.16 | 0.18 | 57.66 | 0.72 |
| adhE2- | 16.93 | 0.0 | 2.67 | 0.88 | 1.61 | 1.98 | 0.12 | 1.01 | 0.67 |
| aor1(Cau) | 12.44 | 0.60 | 0.18 | 0.78 | 2.61 | 1.72 | 0.14 | 4.51 | 0.74 |
| adhE2- | 47.18 | 0.0 | 3.68 | 0.74 | 11.56 | 4.29 | 0.09 | 3.59 | 0.33 |
| aor2(Cau) | 45.28 | 1.49 | 0.0 | 0.0 | 7.06 | 8.40 | 0.19 | inf | 0.98 |
| adhE2- | 57.70 | 0.0 | 3.78 | 0.79 | 13.74 | 4.20 | 0.07 | 3.93 | 0.28 |
| aor3(Cau) | 55.92 | 4.07 | 0.70 | 0.62 | 9.90 | 10.45 | 0.19 | 15.42 | 0.75 |
| adhE2- | 39.00 | 0.0 | 3.29 | 0.77 | 8.61 | 3.68 | 0.09 | 3.03 | 0.37 |
| aor1(Cr) | 21.00 | 1.78 | 0.21 | 0.41 | 2.95 | 3.13 | 0.15 | 9.81 | 0.72 |
| adhE2- | 50.98 | 0.0 | 3.83 | 0.0 | 14.48 | 2.64 | 0.05 | 4.47 | 0.14 |
| aor3(Cr) | 48.78 | 4.36 | 0.40 | 0.0 | 9.31 | 7.08 | 0.15 | 40.98 | 0.50 |
| adhE2- | 50.36 | 0.0 | 4.60 | 1.06 | 9.69 | 5.08 | 0.10 | 2.61 | 0.43 |
| aor4(Cr) | 49.85 | 1.81 | 0.70 | 0.74 | 7.82 | 9.00 | 0.18 | 11.68 | 0.94 |

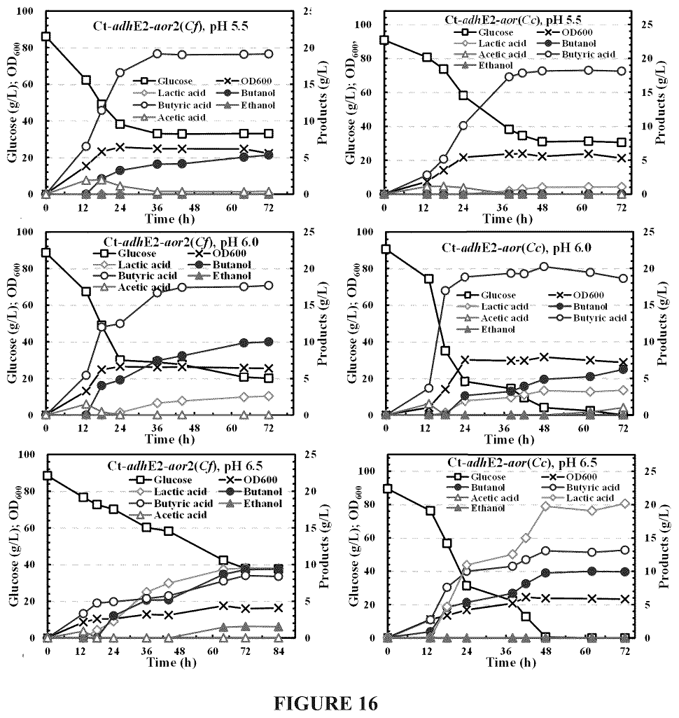

AOR is reversible and the reaction direction can depend on the pH. Therefore, the study investigated the effects of pH on butanol production of two mutants Ct-adhE2-aor2 (Cf) and Ct-adhE2-aor (Cc) that showed significantly increased or reduced butanol production, respectively in bioreactors with pH controlled at 5.5, 6.0, and 6.5 (Table 7 and FIG. 16). Both Ct-adhE2-aor2 (Cf) and Ct-adhE2-aor (Cc) produced more butanol with a higher yield at pH 6.5. In general, all acetic acid was reassimilated, butyric acid production decreased while lactic acid production increased with increasing the pH from 5.5 to 6.5. At pH 6.5, the mutant Ct-adhE2-aor2 (Cf) produced ˜9.4 g/L butanol at the yield of 0.18 g/g glucose, whereas Ct-adhE2-aor (Cc) produced ˜10 g/L butanol but at a lower yield of 0.11 g/g glucose. The increased butanol production with decreased butyric acid and acetic acid production with increased pH may be attributed to increased AOR activities in converting acids to aldehydes.

TABLE 7
Fermentation kinetics of C. tyrobutyricum expressing AOR genes from selected acetogenic
clostridia in bioreactors with pH controlled at 5.5, 6.0, and 6.5, respectively.

| | | Glucose | Lactic | Acetic | | Butyric | | Butanol | | Alcohol/ |
|---|---|---|---|---|---|---|---|---|---|---|
| | | consumed | acid | acid | Ethanol | acid | Butanol | Yield | C4/C2 | Acid |
| Strain | pH | (g/L) | (g/L) | (g/L) | (g/L) | (g/L) | (g/L) | (g/g) | (g/L) | (g/g) |
| Ct-adhE2-aor2 | 5.5 | 52.93 | 0.00 | 0.37 | 0.0 | 19.11 | 5.34 | 0.10 | 66.6 | 0.27 |
| (Cf) | 6.0 | 68.48 | 10.40 | 0.0 | 0.0 | 17.67 | 9.99 | 0.15 | inf | 0.36 |
| | 6.5 | 50.86 | 9.47 | 0.0 | 1.53 | 8.37 | 9.38 | 0.18 | 11.6 | 0.61 |
| Ct-adhE2-aor | 5.5 | 60.35 | 4.27 | 0.0 | 0.0 | 18.11 | 0.0 | 0.0 | inf | 0.00 |
| (Cc) | 6.0 | 90.54 | 13.51 | 1.00 | 0.0 | 18.60 | 6.20 | 0.07 | 24.7 | 0.19 |
| | 6.5 | 89.42 | 20.16 | 0.00 | 0.0 | 13.17 | 9.90 | 0.11 | inf | 0.30 |

In summary, by increasing the available intracellular reducing equivalent NAD(P)H via either overexpressing NADPH-dependent hbd or adding an artificial electron carrier such as MV, engineered C. tyrobutyricum strains can convert glucose, xylose, lactate, and acetate to butanol at a yield of >0.3 g/g in fermentation. With aor or car, butyrate can be reduced to butyraldehyde leading to additional butanol production by ADH in C. tyrobutyricum at a higher butanol yield of >0.4 g/g glucose. Because CO₂ released in the C. tyrobutyricum fermentation can be captured and converted to formate, which can be converted to acetate through the mixotrophic fermentation and then to butanol, the total butanol yield from the lignocellulose hydrolysate sugars can exceed 0.56 g/g sugar.

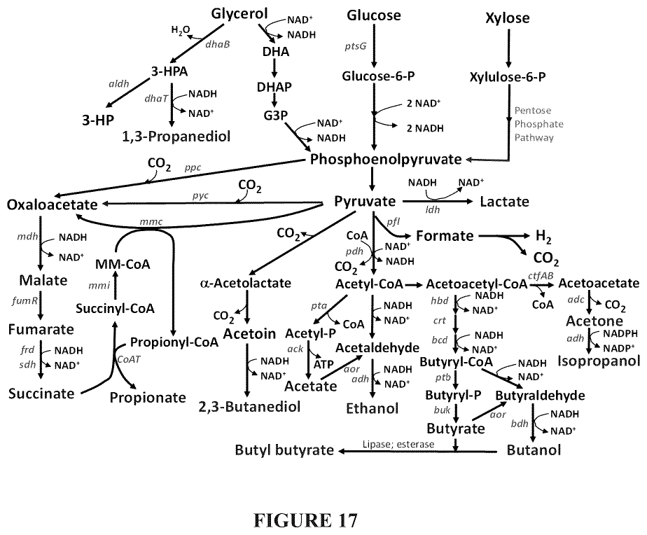

The microbial consortia including LAB, acetogen, and engineered clostridia can also be used to produce other chemicals such as butyric acid, butanediol, propanediol, acetone, isopropanol, butyraldehyde, and esters such as butyl butyrate from various organic feedstocks including cellulose, starch, lactose, sugars, glycerol, and industrial wastes at a significantly increased product yield with little or no CO₂ emission. FIG. 17 illustrates metabolites produced in anaerobes via various metabolic pathways that are either naturally present or can be engineered in Clostridia and other microorganisms such as Escherichia coli.

### REFERENCES

1. Abrini, J., Naveau, H., Nyns, E.-J. (1994) Clostridium autoethanogenum, sp. nov., an anaerobic bacterium that produces ethanol from carbon monoxide. Arch. Microbiol. 161, 345-351.
2. Agarwal A S, Zhai Y, Hill D, Sridhar N, 2011. The electrochemical reduction of carbon dioxide to formate/formic acid: engineering and economic feasibility. ChemSusChem, 4:1301-1310.
3. Bao T, Feng J, Jiang W, Fu H, Wang J, Yang S T (2020) Recent advances in n-butanol and butyrate production using engineered Clostridium tyrobutyricum, World J. Microbiol. Biotechnol. 36:138.
4. Bao, T., Cheng, C., Xin, X., Wang, J., Wang, M., Yang, S T. (2019). Deciphering mixotrophic Clostridium formicoaceticum metabolism and energy conservation: Genomic analysis and experimental studies. Genomics, 111 (6), 1687-1694.
5. Bertsch, J., Mueller, V. (2015) C O metabolism in the acetogen Acetobacterium woodii. Appl. Environ. Microbiol. 81, 5949-5956.
6. Du D, Lan R, Humphreys J, Tao S, (2017) Progress in inorganic cathode catalysts for electrochemical conversion of carbon dioxide into formate or formic acid. J. Appl. Electrochem, 47:661-678
7. Du Y, Jiang W, Yu M, Tang I C, Yang S T (2015) Metabolic process engineering of Clostridium tyrobutyricum Δack-adhE2 for enhanced n-butanol production from glucose: effects of methyl viologen on NADH availability, flux distribution, and fermentation kinetics. Biotechnol Bioeng 112:705-715
8. Fu H, Lin M, Tang I C, Wang J, Yang S T. (2021) Effects of benzyl viologen on increasing NADH availability, acetate assimilation and butyric acid production by Clostridium tyrobutyricum, Biotechnol Bioeng, 118:770-783.
9. Genthner, B., Bryant, M. P. (1982) Growth of Eubacterium limosum with carbon monoxide as the energy source. Appl. Environ. Microbiol. 43, 70-74.
10. Huang, Y., Yang, S. T. (1998). Acetate production from whey lactose using co-immobilized cells of homolactic and homoacetic bacteria in a fibrous-bed bioreactor. Biotechnol Bioeng 60, 498-507.
11. Jones, S. W., Fast, A. G., Carlson, E. D., Wiedel, C. A., Au, J., Antoniewicz, M. R., Papoutsakis, E. T., Tracy, B. P. (2016) CO₂ fixation by anaerobic non-photosynthetic mixotrophy for improved carbon conversion. Nat. Comm. 7:12800.
12. Khusnutdinova A N, Flick R, Popovic A, Brown G, Tchigvintsev A, Nocek B, Correia K, Joo J C, Mahadevan R, Yakunin A F (2017) Exploring bacterial carboxylate reductases for the reduction of bifunctional carboxylic acids. Biotechnol J, 12 (11), 1600751
13. Köpke, M., Held, C., Hujer, S., Liesegang, H., Wiezer, A., Wollherr, A., Ehrenreich, A., Liebl, W., Gottschalk, G., Dürre, P. (2010). Clostridium ljungdahlii represents a microbial production platform based on syngas. PNAS USA 107, 13087-13092.
14. Kopljar D, Inan A, Vindayer P, Wagner N, Klemm E (2014) Electrochemical reduction of CO₂ to formate at high current density using gas diffusion electrodes. J Appl Electrochem, 44:1107-1116.
15. Lee J, Jang Y S, Han M J, Kim J Y, Lee S Y (2016) Deciphering Clostridium tyrobutyricum metabolism based on the whole-genome sequence and proteome analyses. mBio 7 e00743-16
16. Li J, Du Y, Bao T, Dong J, Lin M, Shim H, Yang S T (2019) n-Butanol production from lignocellulosic biomass hydrolysates without detoxification by Clostridium tyrobutyricum Δack-adhE2 in a fibrous-bed bioreactor. Bioresour Technol 289:121749
17. Liew, F., Henstra, A. M., Köpke, M., Winzer, K., Simpson, S. D., Minton, N. P. (2017). Metabolic engineering of Clostridium autoethanogenum for selective alcohol production. Metab Eng 40, 104-114.
18. Nissen L. S., Basen M. (2019) The emerging role of aldehyde: ferredoxin oxidoreductases in microbially catalyzed alcohol production, J Biotechnol 306, 105-117.
19. Poehlein, A., Cebulla, M., Ilg, M. M., Bengelsdorf, F. R., Schiel-Bengelsdorf, B., Whited, G., Andreesen, J. R., Gottschalk, G., Daniel, R., Duerre, P. (2015) The complete genome sequence of Clostridium aceticum: a missing link between rnf- and cytochrome-containing autotrophic acetogens. Mbio. 6 (5), e01168-15.
20. Rumayor M, Dominguez-Ramos A, Irabien A (2018) Formic acid manufacture: Carbon dioxide utilization alternatives, Appl. Sci. 8, 914.
21. Song, A. A. L., In, L. L., Lim, S. H. E., Rahim, R. A. (2017). A review on Lactococcus lactis: from food to factory. Microbial Cell Factories, 16, 55.
22. Tang, I. C., Yang, S. T., Okos, M. R. (1988). Acetic acid production from whey lactose by the co-culture of Streptococcus lactis and Clostridium formicoaceticum. Appl Microbiol Biotechnol 28, 138-143.
23. Wang, S., Huang, H., Moll, J., Thauer, R. K. (2010) NADP⁺ Reduction with reduced ferredoxin and NADP⁺ reduction with NADH are coupled via an electron-bifurcating enzyme complex in Clostridium kluyveri. J Bacteriol 192, 5115-5123.
24. Winkler M (2018) Carboxylic acid reductase enzymes (CARs), Current Opinion in Chemical Biology 43, 23-29
25. Yishai O, Lindner S N, Gonzalez de la Cruz J, Tenenboim H, Bar-Even A (2016) The formate bio-economy. Curr Opin Chem Biol, 35:1-9.
26. Younesi, H., Najafpour, G., Mohamed, A. R. (2005) Ethanol and acetate production from synthesis gas via fermentation processes using anaerobic bacterium, Clostridium ljungdahlii. Biochem. Eng. J. 27, 110-119.
27. Yu M. Zhang Y. Tang I C. Yang S T (2011) Metabolic engineering of Clostridium tyrobutyricum for n-butanol production. Metab Eng 13:373-382

1. A method for converting a carbohydrate-containing source to a carbonaceous product comprising:
contacting a first portion of the carbohydrate-containing source with a first inoculant comprising a lactic acid bacterium (LAB) and an acetogen, thereby forming a first fermentation mixture, wherein the carbohydrate-containing source comprises a carbohydrate, lactate, and formate;
incubating the first fermentation mixture under conditions effective to produce acetate from the carbohydrate, lactate and formate;
contacting a second portion of the carbohydrate-containing source with acetate and a second inoculant comprising a solventogenic Clostridia, thereby forming a second fermentation mixture; and
incubating the second fermentation mixture under conditions effective to produce the carbonaceous product.

2. (canceled)

3. The method of claim 1, wherein the LAB comprises Lactococcus lactis, Streptococcus lactis, Lactococcus plantarum, Lactobacillus delbrueckii, and Lactobacillus bulgaricus, or a combination thereof.

4. The method of claim 1, wherein the acetogen comprises Clostridium formicoaceticum, Acetobacterium woodii, Clostridium aceticum, Clostridium ragsdalei, Clostridium thermocellum, Clostridium autoethanogenum, Clostridium ljungdahlii, Moorella thermoacetica, Eubacterium limosum, or a combination thereof.

5. The method of claim 1, wherein the solventogenic Clostridia comprises Clostridium tyrobutyricum, Clostridium acetobutylicum, Clostridium beijerinkii, Clostridium saccharolyticum, Clostridium carboxidivorans, Clostridium saccharoperbutylacetonicum, Clostridium butylicum, Clostridium cellulovorans, or a combination thereof.

6. (canceled)

7. The method of claim 1, wherein the carbonaceous product comprises n-butanol.

8. The method of claim 1, wherein the carbohydrate-containing source comprises one or more monosaccharides.

9. The method of claim 8, wherein the one or more monosaccharides comprise glucose, xylose, or a combination thereof.

10. The method of claim 1, wherein the carbohydrate-containing source comprises a lignocellulose-derived sugar.

11. The method of claim 1, further comprising adding formate to the first fermentation mixture during the first fermentation.

12. The method of claim 1, wherein incubation of the second fermentation mixture generates CO₂ and H₂ as byproducts, and wherein the method further comprises electrochemically reducing the CO₂ to produce recycled formate.

13. The method of claim 12, wherein the method further comprises adding the recycled formate to the first fermentation mixture.

14. (canceled)

15. (canceled)

16. (canceled)

17. The method of claim 1, wherein at least one of the lactic acid bacteria, the acetogen, and the solventogenic Clostridia comprises a genetically modified bacterium.

18. The method of claim 1, further comprises contacting the second fermentation mixture with methyl viologen (MV), benzyl viologen (BV), neutral red, or a combination thereof.

19. The method of claim 1, wherein the solventogenic Clostridia comprises a Clostridium tyrobutyricum engineered to overexpress an aldehyde/alcohol dehydrogenase (adhE2) gene.

20. The method of claim 1, wherein the solventogenic Clostridia comprises a Clostridium tyrobutyricum engineered to overexpress an NADPH-dependent beta-hydroxybutyryl-CoA dehydrogenase (hbd) gene.

21. The method of claim 1, wherein the acetogen comprises a carboxydotroph.

22. (canceled)

23. The method of claim 1, wherein the LAB comprises a homolactic acid bacterium.

24. (canceled)

25. (canceled)

26. The method of claim 1, wherein the acetate produced from the incubation of the first fermentation mixture is used during the incubation of the second fermentation mixture.

27. A fermentation inoculant for converting a carbohydrate-containing source to a carbonaceous product, the inoculant comprising a lactic acid bacterium (“LAB”) expressing an enzyme that catalyzes the production of lactate from the carbohydrate-containing source, a mixotrophic acetogen expressing an enzyme that catalyzes the production of acetate from lactate and formate, and a solventogenic Clostridia expressing an enzyme that catalyzes the production of the carbonaceous product from the carbohydrate-containing source and acetate.

28. (canceled)

29. (canceled)

30. (canceled)

31. (canceled)

32. (canceled)

33. (canceled)

34. (canceled)

35. (canceled)

36. A system for converting a carbohydrate-containing source to a carbonaceous product, the system comprising:
one or more bioreactors comprising the fermentation inoculant of claim 27 and the carbohydrate-containing source.

37. (canceled)

38. (canceled)

39. (canceled)
